(19) United States
(12) Patent Application Publication
(10) Pub. No.: US 2024/0402164 A1
(43) Pub. Date: Dec. 5, 2024

(54) PLASMONIC CAGED NANOPLATFORMS AND METHODS THEREOF

(71) Applicant: Duke University, Durham, NC (US)

(72) Inventors: Tuan Vo-Dinh, Durham, NC (US); Aidan Canning, Durham, NC (US)

(21) Appl. No.: 18/677,255

(22) Filed: May 29, 2024

Related U.S. Application Data
(60) Provisional application No. 63/605,008, filed on Dec. 1, 2023.
(60) Provisional application No. 63/470,320, filed on Jun. 1, 2023.

(51) Int. Cl. G01N 33/543; G01N 33/53; G01N 33/531

(52) U.S. Cl. CPC G01N 33/54346; G01N 33/5308; G01N 33/531

(57) ABSTRACT

Methods are provided for making caged gold nanostars (C-GNSs). In one method, a layer of gold is deposited on the silver layer of bimetallic nanostar (BNS) particles in a galvanic replacement-free reaction and the silver is subsequently removed via hydrogen peroxide etching. In another method, gold ions are exchanged with the silver atoms of the BNS particles in a galvanic replacement reaction. Both methods result in a hollow gold shell around a gold nanostar core that enables loading with dyes for in vitro and in vivo detection of the C-GNSs and provides an internal standard in sensing. The C-GNS particles have a greater local electric field enhancement from the visible to the NIR spectral range relative to plasmonic-active GNSs of similar diameter that lack the hollow gold shell. Dye-loaded C-GNS particles are demonstrated for in vivo hyperspectral imaging and as photothermal transducers in the treatment of solid tumors.

### CROSS-REFERENCE TO RELATED APPLICATIONS

This application claims priority to U.S. Provisional Patent Application No. 63/470,320, filed on Jun. 1, 2023, and U.S. Provisional Patent Application No. 63/605,008, filed on Dec. 1, 2023, both of which are incorporated by reference herein in their entireties.

### BACKGROUND

Plasmonic nanoparticles have tunable optical properties. These particles have been integrated into applications for biomarker detection, small molecule identification, and solid tumor identification and treatment. One particular nanoparticle morphology proven to have great utility in diagnostic and treatment applications is the gold nanostar (GNS). A GNS generated using a surfactant-free synthesis method has been successfully utilized in photothermal therapy treatments for bladder cancer and glioblastoma in animal models. Bimetallic nanostars (BNS) have also been synthesized in a surfactant-free method. In the case of the BNS, a silver layer is deposited onto the exposed core of the GNS while the branch tips remain exposed. This silver layer shifts the plasmonic resonance of the particles from the NIR to the visible region, enabling their use in label-free Surface-enhanced Raman spectroscopy (SERS) sensing and detection applications. When integrated with a surface-bound oligonucleotide probe, BNS particles have provided the signal enhancement necessary for the amplification-free detection of miRNA biomarkers in solution, substrate, and optical fiber-based assays. In addition to nanoparticles having a star-based morphology, nano rattles are a gold nanosphere within a hollow golden cube that can be loaded with SERS active molecules. Nano rattles have been used to detect pathogenic RNA in blood lysate samples.

However, a need remains for plasmonic-active nanoparticles that are suitable for use in vivo and that incorporate the highly tunable optical properties of nanostar-based particles with the loadable properties of nanoparticles such as nano rattles.

### SUMMARY

In a first aspect of the invention, a method is provided for making plasmonic-active caged gold nanostars, comprising: dispersing one or more bimetallic nanostars into a basic polyvinylpyrrolidone solution, wherein the one or more bimetallic nanostars comprise a gold nanostar having a spherical core, a plurality of branches protruding out from the core, and a layer of silver on the core and extending outward onto the branches, wherein a tip of the branches are exposed gold; and performing one of the following two reactions: i) exchanging gold ions with silver atoms of the bimetallic nanostar in a galvanic replacement reaction, or ii) depositing gold ions to coat a silver surface of the bimetallic nanostars with a layer of gold in a galvanic replacement-free reaction, thereby forming one or more gold-coated bimetallic nanostars, and redispersing the one or more gold-coated bimetallic nanostars into a second solution of hydrogen peroxide to etch the gold layer and remove the silver, thereby forming one or more plasmonic-active caged gold nanostars having a hollow gold shell surrounding the essentially spherical core.

The method can further comprise, optionally, mixing the one or more plasmonic-active caged gold nanostars in a third solution with a compound to be loaded into the hollow gold shell, and dispersing the plasmonic-active caged gold nanostars in a fourth solution and adding gold ions to coat the plasmonic-active caged gold nanostars with a final layer of gold, thereby forming plasmonic-active gold-coated caged gold nanostars and, optionally, having the compound encapsulated within the hollow gold shell, wherein a thickness of the gold shell is essentially uniform and tunable, and wherein the plasmonic-active caged gold nanostars have a tunable plasmon resonance from the visible to the near-infrared (NIR) spectral range.

In another embodiment, the method can further comprise optionally, mixing the one or more plasmonic-active caged gold nanostars in a third solution with a compound to be loaded into the hollow gold shell, and dispersing the plasmonic-active caged gold nanostars in a fourth solution and adding silver ions to coat the plasmonic-active caged gold nanostars with a layer of silver, thereby forming plasmonic-active caged silver-coated gold nanostars and, optionally, having the compound encapsulated within the gold and silver-coated hollow shell, wherein a thickness of the silver-coated gold shell is essentially uniform and tunable, and wherein the plasmonic-active silver-coated caged gold nanostars have a tunable plasmon resonance from the visible to the near-infrared (NIR) spectral range.

In a second aspect of the invention, plasmonic-active gold-coated caged gold nanostars (C-GNS) are provided. The gold-coated C-GNS include: one or more C-GNS having a hollow gold shell surrounding an essentially spherical core and a plurality of gold branches protruding out from the core, wherein a thickness of the gold shell is essentially uniform and tunable. The one or more C-GNS are essentially free of surfactant, have a tunable plasmon resonance ranging from the visible to the near-infrared (NIR) spectral range, and optionally, comprise one or more compounds encapsulated within the hollow gold shell. The one or more compounds can be a dye or a drug.

In a third aspect of the invention, plasmonic-active silver-coated caged gold nanostars (C-GNS) are provided. The silver-coated C-GNS include: one or more C-GNS having a hollow gold shell coated with silver surrounding an essentially spherical core and a plurality of gold branches protruding out from the core, wherein a thickness of the silver-coated gold shell is essentially uniform and tunable. In addition, the one or more silver-coated C-GNS are essentially free of surfactant and have a tunable plasmon resonance ranging from the visible to the near-infrared (NIR) spectral range. Optionally, the silver-coated C-GNS include one or more compounds encapsulated within the hollow silver-coated gold shell. The one or more compounds can be a dye or a drug.

In a fourth aspect of the invention, a method is provided for delivery of a compound to a local environment, comprising: administering to a subject a plasmonic-active caged gold nanostar having one or more compounds loaded within the hollow gold shell, wherein the plasmonic-active caged gold nanostar particles travel to the local environment, and applying laser excitation to the local environment to open the hollow gold shell of the nanostar particles, thereby delivering the compound to the local environment. The local environment can be a local tumor environment and the particles can travel to the local tumor environment within the subject through an enhanced permeability effect and retention effect (EPR). In another aspect, the C-GNS can comprise a bioreceptor that targets the C-GNS to the local environment. The one or more compounds can comprise one or both of an optical reporter and a chemotherapeutic. The method can further include detecting the plasmonic-active caged gold nanostars in the local environment through detection of the optical reporter. The administering can be via oral delivery, intra-arterial injection, or intravenous injection.

In a fifth aspect of the invention, a method is provided for in vitro detection of a target of interest, comprising: contacting one or more plasmonic-active caged gold nanostar particles with a sample, wherein the one or more plasmonic-active caged gold nanostar particles further comprises a bioreceptor for one or more of a cell, a nucleic acid, or a protein target of interest and, optionally, an optical reporter; and detecting an optical signal from one or both of the optical reporter and the one or more compounds loaded within the hollow gold shell. In the method, the bioreceptor can comprise a nucleotide sequence, an aptamer, an antibody, an enzyme, or a cell-based receptor. The optical signal can be a Raman signal or a surface-enhanced Raman scattering (SERS) signal.

In a sixth aspect of the invention, a method is provided for in vivo imaging, comprising: administering to a subject one or more C-GNSs, wherein one or more compounds loaded within the hollow gold shell comprises an optical reporter, and detecting an optical signal from the optical reporter. In the method, the one or more C-GNSs can include a bioreceptor to target the C-GNSs to a cell, protein, or nucleic acid of interest, the bioreceptor optionally comprising a second optical reporter, and the method optionally further comprising detecting a second optical signal from the second optical reporter when the bioreceptor is in the presence of the target of interest.

In a seventh aspect of the invention, a method is provided for in vivo photothermal therapy. The method comprises: administering to a subject one or more C-GNS, wherein the one or more C-GNSs travel to the local environment of interest; and irradiating the local environment of interest to generate an increase in NIR absorption by the particles, thereby increasing the temperature of the local environment of interest. The C-GNS can include one or an optical reporter loaded within the hollow gold shell. The local environment of interest can be a local tumor environment, and the one or more C-GNSs can travel to the local tumor environment through an enhanced permeability and retention effect (EPR). Alternatively, the one or more C-GNSs can include a bioreceptor to target the C-GNSs to a cell or protein of interest, the bioreceptor optionally comprising a second optical reporter, and the method optionally further comprising detecting a second optical signal from the second optical reporter when the bioreceptor is in the presence of the target of interest.

In an eighth aspect of the invention, an automated method is provided for producing plasmonic-active gold nanostars. The method comprises adding to a single reaction vessel with rapid mixing, predetermined timing, and in sequential order the following reagents: (i) an acidic water solution, (ii) a gold ion solution, (iii) a plurality of gold nanospheres, (iv) a silver ion solution, and (v) an ascorbic acid solution, to trigger branch formation on a surface of the gold nanospheres, thereby forming plasmonic-active gold nanostars. The method can further comprise adding a thiolated polyethylene glycol solution to the formed plasmonic-active gold nanostars.

In a ninth aspect of the invention, a system is provided for producing plasmonic-active gold nanostars, comprising: (a) at least one reaction vessel; (b) a reagent dispensing module comprising a microcontroller and at least one motor; and (c) software to execute a method comprising adding to a single one of the at least one reaction vessels with rapid mixing, predetermined timing, and in sequential order the following reagents: (i) an acidic water solution, (ii) a gold ion solution, (iii) a plurality of gold nanospheres, (iv) a silver ion solution, and (v) an ascorbic acid solution, to trigger branch formation on a surface of the gold nanospheres, thereby forming plasmonic-active gold nanostars.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1A is a representative STEM-HAADF image of a typical GNS used as an initial building block of a caged gold nanostar (C-GNS) particle.

FIG. 1B is a STEM-EDS image showing the overlay of the distribution of silver and gold throughout a representative bimetallic nanostar (BNS) particle.

FIG. 1C is a STEM-EDS image of the elemental overlay from a gold-coated BNS particle.

FIG. 1D is a STEM-EDS image of the gold-only channel from a gold-coated BNS particle.

FIG. 1E is a STEM-EDS image of the gold and silver channel overlay from an unsealed C-GNS.

FIG. 1F is a STEM-HAADF image corresponding to FIG. 1E.

FIG. 1G is a STEM-HAADF image showing the high efficiency of C-GNS formation where all BNS that enter the galvanic replacement-free gold coating process result in C-GNS.

FIG. 1H is a plot showing the normalized absorption spectra of each nanoparticle solution GNS, BNS, gold coated BNS, and unsealed C-GNS.

FIG. 1I is a plot showing the heat losses spectra for both particle models GNS and open C-GNS.

FIG. 1J is a depiction of the normalized electric field |E/|/Eo| around the resonance peak labeled i in FIG. 1I corresponding to the tips of the GNS branches.

FIG. 1K is a depiction of the normalized electric field |E|/|Eo| around the resonance peak labeled ii in FIG. 1I corresponding to the tips of the C-GNS branches.

FIG. 1L is a depiction of the normalized electric field |E|/|Eo| around wavelength labeled iii in FIG. 1I, which represents the interaction between the C-GNS model and 900 nm light.

FIG. 2A is a STEM-EDS image of representative gold-coated C-GNS from the AU_1 batch.

FIG. 2B is a STEM-EDS image of representative gold-coated C-GNS from the AU_3 batch.

FIG. 2C is a STEM-EDS image of representative gold-coated C-GNS from the AU_5 batch.

FIG. 2D is a STEM-EDS overlay image corresponding to FIG. 2A.

FIG. 2E is a STEM-EDS overlay image corresponding to FIG. 2B.

FIG. 2F is a STEM-EDS overlay image corresponding to FIG. 2C.

FIG. 2G is a brightfield STEM image providing improved internal nanoparticle contrast as the shell thickness increases compared to HAADF.

FIG. 2H is a plot showing normalized absorbance as a function of wavelength for each of AU_1, AU_2, AU_3, AU_4, and AU_5 batches.

FIG. 2I is a plot showing SERS intensity as a function of wavenumber for each of AU_1, AU_2, AU_3, AU_4, and AU_5 batches.

FIG. 2J is two plots showing SERS intensity as a function of wavenumber for unsealed and sealed C-GNS batch AU_3 after incubation with HITC dye and subsequent addition of hydrogen peroxide at a number of different time frames.

FIG. 3A is a schematic showing the upper right quadrant of a C-GNS model cross-section with the swept dimensions indicated with red arrows.

FIG. 3B is a scatter plot showing the internal heat losses result of all 192 simulated C-GNS model variants.

FIG. 3C is a model of a C-GNS that produced the highest internal heat losses value, with a branch length of 35 nm, a branch tip to inner shell edge distance of 5 nm, and a shell thickness of 5 nm.

FIG. 3D illustrates the normalized electric field |E|/|Eo| for the entire particle model and only the interior water cavity area.

FIG. 3E illustrates the normalized electric field |E|/|Eo| for the entire particle model and only the interior water cavity area and shows the area of greatest enhancement occurs in the ring-shaped region at the junction of the branch and shell.

FIG. 3F contains the interior water cavity's cross-section of the particle model, illustrating significant enhancement propagating along the branch.

FIG. 3G is a schematic showing the |E|/|Eo| map of the models which generated the greatest internal heat losses values with a branch tip to inner shell edge distance of 5, 10, and 15 nm, respectively, from left to right.

FIG. 4A is a schematic showing the total emission of the agarose gel phantom C-GNS when excited with 785 nm light.

FIG. 4B shows direct images of the phantom of FIG. 4A at 820, 822, and 824 nm wavelengths in ascending order.

FIG. 4C is a graph showing reference spectra of HITC dye in C-GNS, where the labels i, ii, and iii correspond to the bottom, middle, and top slices of FIG. 4B.

FIG. 4D is an image showing a mouse 24 hours after nanoparticle injection, with the tumor in the highlighted region.

FIG. 4E is a bright field image of the tumor region of the mouse in FIG. 4D through the lab-built microscope system.

FIG. 4F is a hyperspectral image of the same region of FIG. 4E at 822 nm.

FIG. 4G is a bright field image of the photothermal treatment of a mouse that had received C-GNS shown in FIG. 4D.

FIG. 4H is an IR camera image of the photothermal treatment of a mouse that had received C-GNS shown in FIG. 4D.

FIG. 4I is a graph showing the temperature results of a hypodermic thermocouple placed into the center of the tumor as a function of time for each of six mice treated with 450 mW/cm2 1064 nm light, where three received an injection of 1.5 mg C-GNS, and three did not.

FIG. 4J is a graph showing the maximum values of each thermal camera measurement for the control and C-GNS treatment groups of mice from FIG. 4I.

FIG. 5A is a schematic of a plasmonic caged nanostar (C-GNS) based on a gold nanostar with a dye label(s) and/or drugs in the interstitial gap.

FIG. 5B is a schematic of the plasmonic caged nanostar (C-GNS) of FIG. 5A with multimodal labels (MRI, CT, PET, etc.) on the outside shell.

FIG. 5C is a schematic of a plasmonic caged nanostar (C-GNS) based on a gold nanocube core.

FIG. 5D is a schematic of a plasmonic caged nanostar (C-GNS) based on a gold nanotriangle core.

FIG. 5E is a schematic of a plasmonic caged nanostar (C-GNS) based on a gold nanostar with a paramagnetic spherical inner core.

FIG. 5F is a schematic of a plasmonic caged nanostar (C-GNS) based on a gold nanostar with an elongated paramagnetic inner core.

FIG. 5G is a schematic of a plasmonic caged nanostar (C-GNS) based on a gold nanostar labeled with multimodal labels with a protective coating as needed.

FIG. 6A is a schematic of a plasmonic caged nanostar (C-GNS) based on a gold nanostar with bioreceptors with a dye label(s) and/or drugs in the interstitial gap.

FIG. 6B is a schematic of the plasmonic caged nanostar (C-GNS) of FIG. 6A with multimodal labels (MRI, CT, PET, etc.) on the outside shell.

FIG. 6C is a schematic of a plasmonic caged nanostar (C-GNS) based on a gold nanocube core with bioreceptors.

FIG. 6D is a schematic of a plasmonic caged nanostar (C-GNS) based on a gold nanotriangle core with bioreceptors.

FIG. 6E is a schematic of a plasmonic caged nanostar (C-GNS) based on a gold nanostar with a paramagnetic spherical inner core with bioreceptors.

FIG. 6F is a schematic of a plasmonic caged nanostar (C-GNS) based on a gold nanostar with an elongated paramagnetic inner core with bioreceptors.

FIG. 6G is a schematic of a plasmonic caged nanostar (C-GNS) based on a gold nanostar with bioreceptors labeled with multimodal labels with a protective coating as needed.

FIG. 7A is a schematic of a plasmonic caged nanostar (C-GNS) having an antibody (or nanobody) bioreceptor that captures a target antigen and exhibits SERS when the antigen gets close to the metal surface of the nanostar. The C-GNS provides an internal standard SERS signal.

FIG. 7B is a schematic of a plasmonic caged nanostar (C-GNS) having an Inverse Molecular Sentinel (iMS) probe to hybridize with a target nucleic acid (DNA, mRNA, miRNA) which displaces the capture probe such that the iMS forms a stem-loop structure leading to a SERs signal. The C-GNS provides an internal standard SERS signal.

FIG. 7C is a schematic of a plasmonic caged nanostar (C-GNS) having an aptamer probe to capture a target protein or small molecule which displaces the capture probe such that the Raman label is located closer to the nanostar surface, leading to a strong SERs signal. The C-GNS provides an internal standard SERS signal.

FIG. 8A is a schematic illustrating the plasmonic coupling detection concept for nucleic acid targets consisting of two C-GNSs each having a SERS dye embedded inside the interstitial core-shell gap and each having a probe DNA sequence identical to one half of the target sequence of interest and a Raman label at the end of each probe DNA sequence.

FIG. 8B is a schematic illustrating that when the two types of C-GNSs of FIG. 8A are mixed with the target sequence, they hybridize to the target probe in such a way that the SERS labels are in the middle resulting in an increase of the SERS signal intensities of the two Raman labels.

FIG. 9A is a schematic illustrating the plasmonic coupling detection concept for protein targets consisting of two C-GNSs each having a SERS dye embedded inside the interstitial core-shell gap and each having a having a bioreceptor (e.g., capture antibody target to specific protein or a cancer cell) and a Raman label attached to the bioreceptor.

FIG. 9B is a schematic illustrating that when the two types of C-GNSs of FIG. 9A are mixed with the target protein, they bind to the target protein in such a way that the SERS labels are in the middle resulting in an increase of the SERS signal intensities of the two Raman labels.

FIG. 10A is a schematic illustrating a C-GNS C-GNS sensor having a SERS dye embedded inside the interstitial core-shell gap and having a probe DNA sequence identical to one half of the target sequence of interest attached thereto (Capture Probe 1) and a magnetic bead having an attached second probe DNA sequence (Capture Probe 2) identical to the other half of the target.

FIG. 10B is a schematic illustrating the C-GNS and the magnetic particle of FIG. 10A with the capture probe sequences hybridized to the target sequence.

FIG. 10C is a schematic illustrating the operating principle of using magnetic beads to concentrate the C-GNS sensors of FIG. 10B for detection of nucleic acid targets.

FIG. 11A is a schematic illustrating a C-GNS C-GNS sensor having a SERS dye embedded inside the interstitial core-shell gap and having a first capture antibody for a target of interest attached thereto (Capture Antibody 1) and a magnetic bead having an attached second capture antibody (Capture Antibody 2).

FIG. 11B is a schematic illustrating the C-GNS and the magnetic particle of FIG. 11A with the target protein bound to both Capture Antibodies 1 and 2.

FIG. 11C is a schematic illustrating the operating principle of using magnetic beads to concentrate the C-GNS sensors of FIG. 11B for detection of protein targets.

FIG. 12A is a schematic illustrating a C-GNS C-GNS sensor having an attached iMS probe in the “ON” position and a SERS dye embedded inside the interstitial core-shell.

FIG. 12B is a schematic of a multiplex C-GNS system of FIG. 12A with different labels for different targets.

FIG. 13A is a schematic illustrating a C-GNS C-GNS sensor having a magnetic core, an attached iMS probe in the “ON” position, and a SERS dye embedded inside the interstitial core-shell.

FIG. 13B is a schematic of a multiplex C-GNS system of FIG. 13A with different labels for different targets.

FIG. 14A is a STEM-EDS image illustrating C-GNS C-GNSs coated with a first thickness of silver.

FIG. 14B is a STEM-EDS image illustrating the C-GNS C-GNSs of FIG. 14A coated with an increased thickness of silver.

FIG. 14C is a STEM-EDS image illustrating the C-GNS C-GNSs of FIG. 1BA coated with an increased thickness of silver.

FIG. 14D is an image showing the subsequent change in the particle absorption spectra for the C-GNSs of FIG. 14A.

FIG. 14E is an image showing the subsequent change in the particle absorption spectra for the C-GNSs of FIG. 14B.

FIG. 14F is an image showing the subsequent change in the particle absorption spectra for the C-GNSs of FIG. 14C.

FIG. 14G is a graph showing relative absorbance as a function of wavelength for the C-GNSs having variable silver coating thickness.

FIG. 14H is a graph showing SERS intensity as a function of wavenumber for the C-GNSs having variable silver coating thickness.

FIG. 15 shows a conceptual drawing of a C-GNS functionalized substrate integrated into device-based biomarker sensing applications.

FIG. 16 shows an example of the differences between the spectra of the bimetallic nanostars (i.e., no cage) in both the “ON” and “OFF” positions and the bimetallic C-GNS particles (i.e., with cages) in the iMS assay.

FIG. 17 shows the SERS Spectra of C-GNS particles filled with different molecules.

FIG. 18A is a TEM image of a gold-sealed C-GNS particle filled with dye molecules.

FIG. 18B is an image of a gold-sealed C-GNS particle of FIG. 18A after chemical etching demonstrating that via selective chemical etching, the shell of the C-GNS can be removed.

FIG. 18C is an image of gold-sealed C-GNS particles of FIG. 18A which shows selectively opened C-GNS particles where the selective opening was performed with a pulsed laser.

FIG. 19 is a schematic illustrating the basic operating principle of the in vitro diagnostics modality using C-GNS probes.

FIG. 20A is a schematic illustrating the basic operating principle for the in vivo diagnostic and therapy modality using C-GNS probes showing delivery of a C-GNS drug to a person.

FIG. 20B is a schematic illustrating the basic operating principle of the in vivo diagnostic and therapy modality using C-GNS probes showing photothermal therapy and detection in a person.

FIG. 21A is a schematic diagram outlining an automated GNS synthesis method and device.

FIG. 21B is a schematic depicting GNS formation using the method and device of FIG. 21A.

FIG. 21C is an image of the GNS device of FIG. 21A after producing a batch of GNS nanoparticles.

FIG. 22A is a graph showing the injection precision for the volume dispensed 30 times in a row using the GNS synthesis device.

FIG. 22B is a graph showing the absorbance spectra of GNS solutions where the mixing speed was varied.

FIG. 22C is a graph showing the absorbance spectra of base “s10” GNS particle as the amount of gold chloride added to the solution was varied.

FIG. 22D is a graph showing the absorbance spectra of base “s25” GNS particle as the amount of gold chloride added to the solution was varied.

FIG. 22E is a graph showing the absorbance spectra of base “s50” GNS particle as the amount of gold chloride added to the solution was varied.

FIG. 22F is a graph showing the absorbance spectra of base “s50” GNS created using the same amount of gold, while varying the delay between silver nitrate and ascorbic acid addition, N=3.

FIG. 22G is a HAADF STEM image of “s10” particles prepared with 5 mL of gold chloride.

FIG. 22H is a HAADF STEM image of “s50” particles prepared with 5 mL of gold chloride.

FIG. 23 is a schematic diagram outlining an automated method and device for synthesizing bimetallic nanostars where the device comprises an additional motor relative to the method and device of FIG. 21A.

FIG. 24A is a graph showing the absorbance spectra of base GNS morphology “s25_3mLAu” as the amount of silver added increases.

FIG. 24B is a graph showing the absorbance spectra of base GNS morphology “s25_4mLAu” as the amount of silver added increases.

FIG. 24C is a graph showing the absorbance spectra of base GNS morphology “s25_5mLAu” as the amount of silver added increases.

FIG. 24D is a graph showing the absorbance spectra of base GNS morphology “s50_3mLAu” as the amount of silver added increases.

FIG. 24E is a graph showing the absorbance spectra of base GNS morphology “s50_4mLAu” as the amount of silver added increases.

FIG. 24F is a graph showing the absorbance spectra of base GNS morphology “s50_5mLAu” as the amount of silver added increases.

FIG. 24G is a STEM-EDS image of S25_5 mL_dot25Ag particles.

FIG. 24H is a STEM-EDS image of S25_5 mL_1dot25Ag particles.

FIG. 24I is a STEM-EDS image of S25_5 mL_2Ag particles.

FIG. 25A shows the SERS spectra of S25_5 mL particles with a silver addition amount ranging from 0.25 mL to 2 mL when labeled with p-MBA.

FIG. 25B is a graph of SERS intensity as a function of AgNO3 added for the S25_3 mL, S25_4 mL, and S25_5 mL particles at 633 nm.

FIG. 25C is a graph of SERS intensity as a function of AgNO3 added for the S25_3 mL, S25_4 mL, and S25_5 mL particles at 785 nm.

FIG. 25D is a graph of SERS intensity as a function of AgNO3 added for the S50_3 mL, S50_4 mL, and S50_5 mL particles at 633 nm.

FIG. 25E is a graph of SERS intensity as a function of AgNO3 added for the S50_3 mL, S50_4 mL, and 50_5 mL particles at 785 nm.

FIG. 26 is a schematic diagram showing an embodiment of a Massively Parallel AC-GNS system for large-scale production of nanoparticles.

FIG. 27 is a graph of a comparison of the change in extinction spectra between GNS particles and C-GNS particles.

FIG. 28A ICP-MS results showing the gold-to-silver ratio of C-GNS particles based on the preparation method used.

FIG. 28B is a graph showing cytotoxicity study results of particles based on the preparation method used.

FIG. 29A is a HAADF-STEM image of C-GNS particle prepared via galvanic reaction method.

FIG. 29B is a STEM-EDS image of C-GNS particle prepared via galvanic reaction method.

FIG. 29C is a HAADF image of C-GNS particle prepared via galvanic replacement free preparation method.

FIG. 29D is a STEM-EDS image of C-GNS particle prepared via galvanic replacement free preparation method.

FIG. 29E is a quantification of the STEM-EDS image of the particle shown in FIG. 29B.

FIG. 29F is a quantification of the STEM-EDS image of the particle shown in FIG. 29D.

FIG. 29G is a graph showing the change in absorption spectra of C-GNS solution during the galvanic replacement process.

FIG. 29H is a graph showing the change in absorption spectra of C-GNS solution during galvanic replacement-free process.

### DETAILED DESCRIPTION

To promote an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or more than one (i.e., at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements.

As used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.” Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used herein, the terms “hollow gold shell” and “interstitial gap” are interchangeable. As used herein, the terms “plasmonic-active caged gold nanostar,” “plasmonic caged gold nanostar,” “caged gold nanostar,” “caged gold nanostar particles,” “C-GNS,” and “caged gold nanostar nanoparticles,” are interchangeable.

As used herein, the term “plasmonic caged nanostar” or “C-GNS” refers to a plasmonic-active caged gold nanostar or a plasmonic-active caged gold nanostar.

As used herein, the terms “plasmonic-active caged bimetallic nanostar,” “plasmonic bimetallic nanostar,” “bimetallic nanostar,” “BNS, and “bimetallic nanoparticles,” are interchangeable.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered expressly stated in this disclosure. Unless otherwise defined, all technical terms used herein have the same meaning as commonly understood by one of ordinary skill in the art to which this disclosure belongs.

Described herein is a method to unify the highly tunable optical properties of nanostar-based particles with the loading capability of hollow-shell particles such as nano rattles resulting in caged gold nanostars (C-GNSs). As described herein and in the Examples provided herein, the C-GNSs can be synthesized in a method in which the silver layer of the bimetallic nanostar (BNS) particles is used as scaffolding to deposit a layer of gold in a galvanic replacement-free reaction and is subsequently removed via hydrogen peroxide etching. In another method, gold ions are exchanged with the silver atoms of the BNS particles in a galvanic replacement reaction. Both methods result in a hollow gold shell around a gold nanostar core that enables loading with dyes for in vitro and in vivo detection of the C-GNSs and provides an internal standard in sensing. The C-GNS particles have a greater local electric field enhancement from the visible to the NIR spectral range relative to plasmonic-active GNSs of similar diameter that lack the hollow gold shell. In addition, the C-GNS particles have superior thermal stability relative to plasmonic-active GNSs of a similar diameter that lack the hollow gold shell. The C-GNS particles can be further coated with gold or silver to further tune their plasmonic properties from the visible to the near-infrared (NIR) spectral range. The loading ability of the C-GNS particles and the effects of increasing the gold or gold/silver shell thickness on the nanoparticle absorption spectra and SERS signal enhancement are demonstrated herein and described in the Examples herein. Finite Element Method (FEM) simulations of the electric field generated by numerous different C-GNS particle morphologies support the SERS signal enhancement of the C-GNS provided herein. In vivo hyperspectral imaging of dye-loaded C-GNS particles is demonstrated as well as their performance as photothermal transducers in the treatment of solid tumors.

A new platform of plasmonic nanoparticles that combines the adjustable optical properties of nanostars with the loading capacity of traditional spherical or rectangular hollow metallic nanoparticles is described herein. One method of synthesizing the nanoparticles provided herein includes depositing a layer of gold on a silver-coated gold nanostar and then oxidizing the silver layer with hydrogen peroxide to create a hollow shell structure. This approach significantly reduces the amount of gold-silver alloy formation in the caged gold nanostar (C-GNS) shell compared to traditional galvanic replacement, and no ionic surfactant is used in the preparation process. This makes C-GNS particles suitable for applications such as photothermal therapy for cancer treatment. Additionally, the plasmonic coupling between the shell and the branch tips results in a high NIR absorption while maintaining the same particle diameter. Cargo, such as dye, can be effectively loaded and sealed within these particles. Furthermore, the greatest electric field enhancement happens at the junction of the nanostar branch and inner surface of the shell well. Because the C-GNS have complex internal structures, they provide more areas of intense field enhancement compared to other core-hollow-shell particles with less complicated internal structures.

The loadable structure of the caged gold nanostars described herein maintains the tunable optical properties of nanostar-based particles with the added functionality of a loadable region, all without the use of ionic surfactants. As a result, the caged gold nanostars described herein can also have utility in vivo with hyperspectral imaging for tumor localization and photothermal therapy for the ablation of solid tumors.

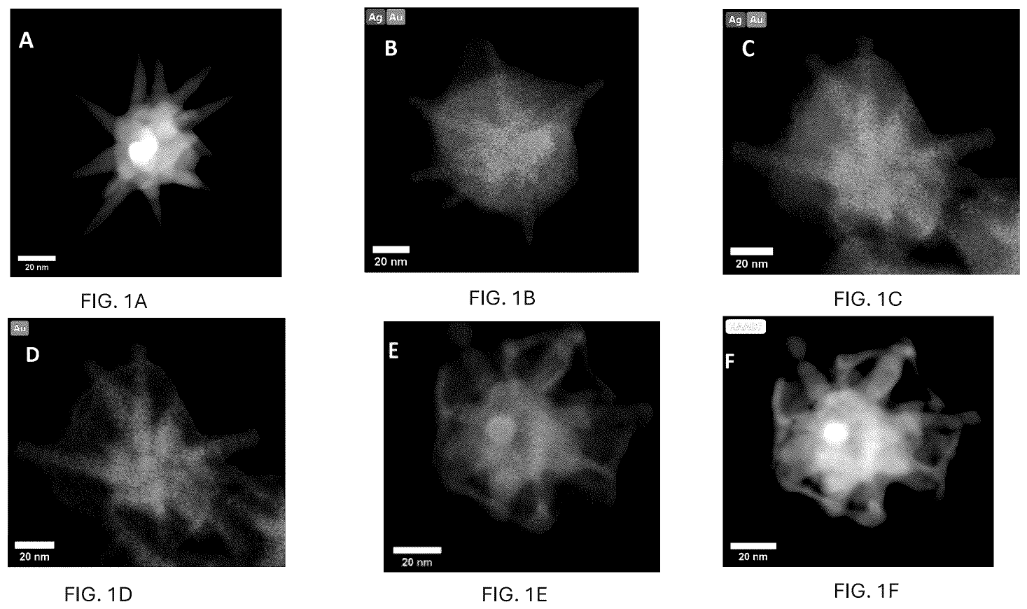

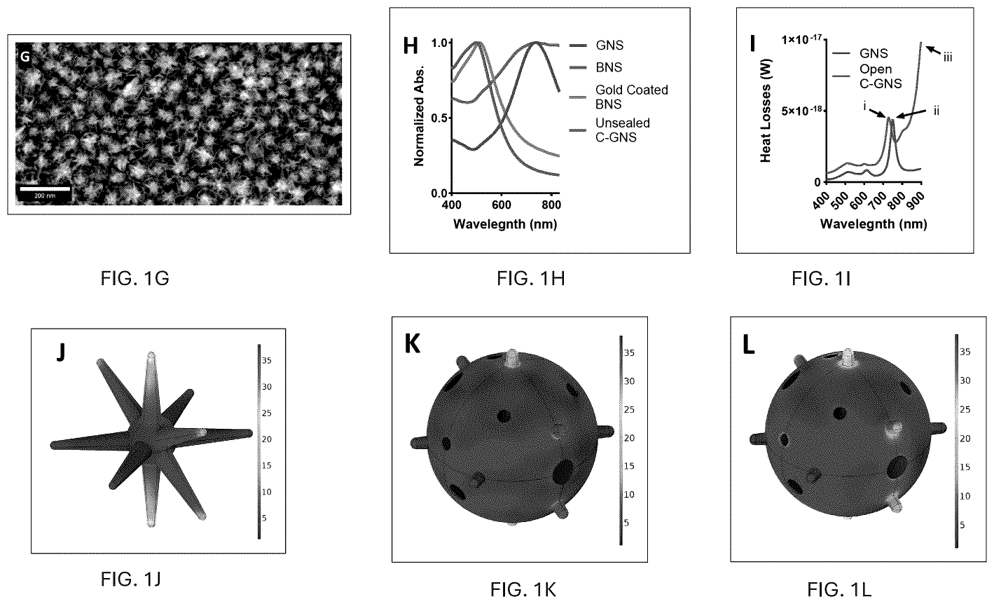

In one embodiment, a galvanic replacement-free method is provided for making the C-GNSs. To initiate C-GNS preparation, GNS are synthesized using a surfactant-free method (see Example 1 and FIGS. 1-20). FIG. 1A contains a representative STEM-HAADF image of a typical GNS used as an initial building block of a C-GNS particle. A layer of silver is then deposited onto the exposed core of the GNS, growing outwards to the branch tips, resulting in a bimetallic nanostar (BNS). A STEM-EDS image showing the overlay of the distribution of silver and gold throughout a representative BNS particle is shown in FIG. 1B. Next, gold is deposited in a galvanic replacement-free reaction to coat the silver of the bimetallic nanostars with a layer of gold, thereby forming gold-coated BNS's. FIGS. 1C and 1D show STEM-EDS images of the elemental overlay and the gold-only channel from a gold-coated BNS particle. While a galvanic replacement reaction would result in the formation of a hollow cage structure, a by-product of that process would be the formation of a gold-silver alloy as the shell.

Gold nanoparticles are attractive vehicles for a wide variety of in vivo applications, one of which is that they have the lowest associated toxicity of all metal nanoparticles. In vivo, silver nanoparticles have been shown to cause greater reactive oxygen species (ROS) production than their gold counterparts. However, in a study examining the biocompatibility of gold, silver, and gold-silver alloy nanoparticles, no significant difference in cell maturation or expansion was observed between gold nanoparticles and 1:1 gold-silver alloy particles. Therefore, the galvanic replacement-free synthetic route results in the lowest amount of gold-silver alloy formation in the shell of the C-GNS.

In the method, the gold-coated BNS particles are redispersed into a second solution of hydrogen peroxide to etch the gold layer and remove the silver, thereby forming one or more plasmonic-active caged gold nanostars having a hollow gold shell surrounding a core, which, in embodiments, can have an essentially spherical shape. This method of hollow shell formation uses the original silver shell as a removable scaffold upon which to build the golden outer shell. The etching process results in an unsealed C-GNS. FIG. 1E contains a STEM-EDS image of the gold and silver channel overlay from an unsealed C-GNS, and FIG. 1F contains the corresponding STEM-HAADF image. The high efficiency of C-GNS formation is seen in the STEM-HAADF image in FIG. 1G, where all BNS that enter the galvanic replacement-free gold coating process result in C-GNS. The plot in FIG. 1H contains the normalized absorption spectra of each nanoparticle solution.

A method is provided for making plasmonic-active caged gold nanostars, comprising: dispersing one or more bimetallic nanostars into a basic polyvinylpyrrolidone solution, wherein the one or more bimetallic nanostars comprise a gold nanostar having a spherical core, a plurality of branches protruding out from the core, and a layer of silver on the core and extending outward onto the branches, wherein a tip of the branches are exposed gold; and performing one of the following two reactions: i) exchanging gold ions with silver atoms of the bimetallic nanostar in a galvanic replacement reaction, or ii) depositing gold ions to coat a silver surface of the bimetallic nanostars with a layer of gold in a galvanic replacement-free reaction, thereby forming one or more gold-coated bimetallic nanostars, and redispersing the one or more gold-coated bimetallic nanostars into a second solution of hydrogen peroxide to etch the gold layer and remove the silver, thereby forming one or more plasmonic-active caged gold nanostars having a hollow gold shell surrounding the essentially spherical core.

The method can further include first forming the one or more bimetallic nanostars for dispersing into the basic polyvinylpyrrolidone solution. The bimetallic nanostars can be formed by depositing a layer of silver onto an exposed core of one or more gold nanostars, each nanostar having a plurality of branches protruding out from the core, wherein the layer of silver grows outward onto the branches and leaves a tip of the branches exposed, thereby forming the one or more bimetallic nanostars. With this method, the plasmonic-active caged gold nanostars are essentially free of surfactant.

The method can further comprise optionally, mixing the one or more plasmonic-active caged gold nanostars in a third solution with a compound to be loaded into the hollow gold shell, and dispersing the plasmonic-active caged gold nanostars in a fourth solution and adding gold ions to coat the plasmonic-active caged gold nanostars with a final layer of gold, thereby forming plasmonic-active gold-coated caged gold nanostars and, optionally, having the compound encapsulated within the hollow gold shell, wherein a thickness of the gold shell is essentially uniform and tunable, and wherein the plasmonic-active gold-coated caged gold nanostars have a tunable plasmon resonance from the visible to the near-infrared (NIR) spectral range.

In another embodiment, the method can further comprise optionally, mixing the one or more plasmonic-active caged gold nanostars in a third solution with a compound to be loaded into the hollow gold shell, and dispersing the plasmonic-active caged gold nanostars in a fourth solution and adding silver ions to coat the plasmonic-active caged gold nanostars with a layer of silver, thereby forming silver-coated plasmonic-active caged gold nanostars and, optionally, having the compound encapsulated within the gold and silver-coated hollow shell, wherein a thickness of the silver-coated gold shell is essentially uniform and tunable, and wherein the plasmonic-active silver-coated caged gold nanostars have a tunable plasmon resonance from the visible to the near-infrared (NIR) spectral range.

In the method where the plasmonic-active caged gold nanostars are formed according to reaction (ii) (i.e., a galvanic replacement-free reaction), the plasmonic-active caged gold nanostars can have a reduced amount of gold-silver alloy in the gold shell compared to plasmonic-active caged gold nanostars formed according to reaction (i) (a galvanic replacement reaction). The reduced amount of the gold-silver alloy in the gold shell can be a reduction of at least 75%.

In the method, the dispersing and exchanging steps, or the dispersing, depositing, and redispersing steps can be automated and can occur in a single reaction vessel with rapid mixing and predetermined timing.

GNS has a tunable plasmon resonance in the NIR, depending on branch length and aspect ratio. The addition of silver onto the surface of the GNS results in a blue shift of the absorbance spectrum. After silver oxidation and removal via hydrogen peroxide etching, the absorbance of the C-GNS particle solution red shifts. There is a noticeable widening of the absorption spectra between GNS and C-GNS solutions. FIGS. 1I-L illustrate a wave optics module to evaluate the structural cause of this widening, with corresponding models for GNS and C-GNS particles. FIG. 1I shows the heat loss spectra for both particle models. Points labeled i, ii, and iii in FIG. 1I correspond to FIGS. 1J-1K, respectively. These figures depict the normalized electric field |E|/|Eo| around the nanoparticle model at the given wavelength. The resonance peaks labeled i and ii in FIG. 1I correspond to the tips of the GNS and C-GNS branches shown in FIGS. 1J and 1K. FIG. 1L, which represents the interaction between the C-GNS model and 900 nm light, shows an intense enhancement in the local electric field. The plasmonic coupling between the particle branches and shell supports the peak broadening in the NIR region of the C-GNS absorption spectra.

Photothermal therapy leverages the high NIR absorption of nanostar-based particles to generate a local temperature increase sufficient to trigger cell death. Particles that have the highest possible NIR absorption are the most attractive for in vivo heating applications because the more efficiently heat is generated, the lower the minimum effective dose becomes.

Traditionally, the peak absorbance of GNS is redshifted by increasing the nanoparticle branch length and aspect ratio. However, the enhanced permeability and retention (EPR) effect, which allows for the passive targeting and accumulation of nanoparticles within the tumor microenvironment, significantly diminishes as the particle diameter increases over 100 nm. Therefore, the C-GNS described herein provide an alternative to having to increase nanoparticle size. The C-GNS have increased absorption in the NIR due to the interaction between the branches and shell compared to GNS of the same size.

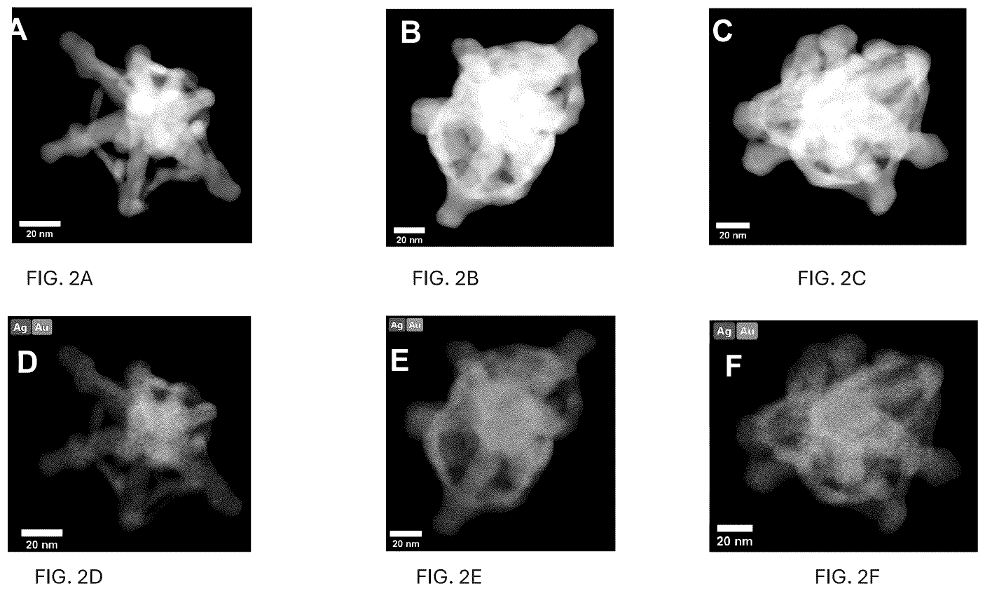

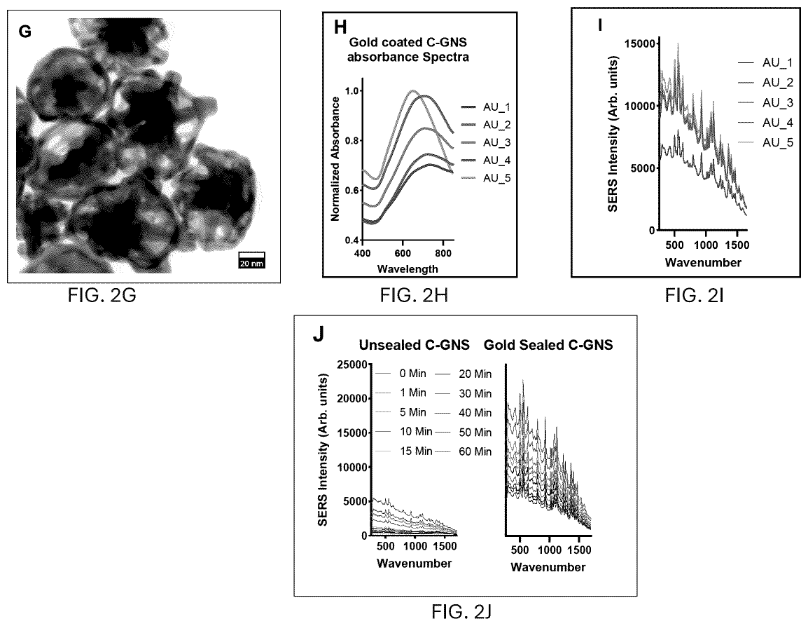

The hollow shell around the C-GNS can be loaded with cargo including but not limited to dyes and drugs. The method of making the C-GNS can further include loading and sealing the C-GNS. The method includes mixing the C-GNSs in a solution with a compound to be loaded into the hollow gold shell, and then adding gold ion to coat the C-GNSs with a final layer of gold, thereby forming gold-coated C-GNSs. FIGS. 2A-2C contain STEM-EDS images of representative gold-coated C-GNS from AU_1, AU_3, and AU_5 batches in which HITC dye was loaded and sealed within the C-GNS. FIGS. 2D-2F are the corresponding STEM-EDS overlay images. The brightfield STEM image in FIG. 2G provides improved internal nanoparticle contrast as the shell thickness increases compared to HAADF.

As the gold layer increases in thickness, the peak in the absorption spectrum blue shifts, while the relative absorbance in the 800 nm range decreases, most noticeably seen in the AU_5 formulation in FIG. 2H. The widening of the branch tips seen in FIGS. 2C and 2F causes this blue shift to occur. SERS measurements of each gold-coated sample show the effect that increasing the thickness of the outer gold layer has on signal intensity. The sample that had received the lowest molar ratio of gold, AU_1, had the lowest SERS signal, shown in FIG. 2I. Samples AU_2,3,4 had very similar intensities, with sample AU_5 having the largest SERS signal. The spectra shown in FIG. 2J show strong evidence that the HITC dye was effectively scaled within the C-GNS and not adsorbed onto the particle surface.

The one or more plasmonic-active caged gold nanostars having a hollow gold shell surrounding the essentially spherical core, produced according to the method described herein, can be loaded with a compound and the compound sealed within the hollow shell. Specifically, the method comprises mixing the one or more plasmonic-active caged gold nanostars in a solution with a compound to be loaded into the hollow gold shell, and dispersing the plasmonic-active caged gold nanostars in another solution and adding gold or silver ions to coat the plasmonic-active caged gold nanostars with a final layer of gold or silver, thereby forming gold- or silver-coated plasmonic-active caged gold nanostars having the compound loaded within the hollow gold shell. The thickness of the gold or silver shell can be essentially uniform and tunable, and the plasmonic-active caged gold nanostars can have a tunable plasmon resonance in the near infrared (NIR).

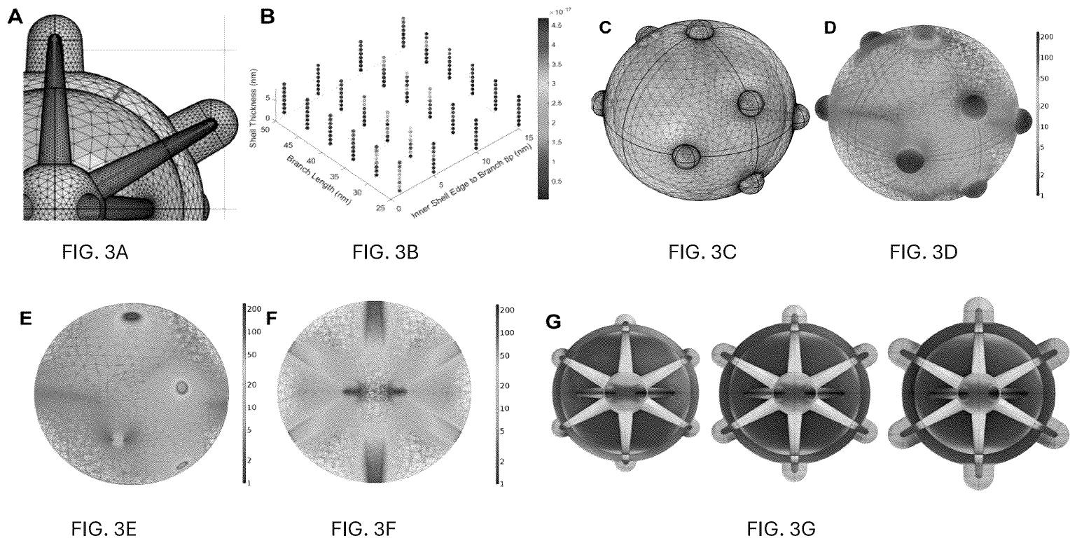

FIG. 3 shows models of C-GNSs created with a wave optics package to investigate the SERS signal enhancement trends seen in FIG. 2. The local electric field enhancement |E|/|Eo| at 785 nm is shown for a wide array of different C-GNS morphologies. Three nanoparticle dimension parameters are varied to encompass the heterogeneity in these C-GNS particles of various gold coating thicknesses. The schematic in FIG. 3A is the upper right quadrant of a C-GNS model cross-section with the swept dimensions indicated with red arrows. Starting from the left, the first arrow indicates the distance between the branch tip and the inner edge of the gold shell, simulated at 15 nm, 10 nm, 5 nm, and 1 nm. The second arrow represents the shell thickness, swept between 1 and 8 nm in thickness in steps of 1 nm. Finally, the third red arrow in FIG. 3A depicts the branch length, simulated at 25 nm, 30 nm, 35 nm, 40 nm, 45 nm, and 50 nm.

The internal heat loss results of all 192 simulated C-GNS model variants are shown in the scatter plot in FIG. 3B. The domain of swept dimensions having the greatest degree of local electric field enhancement is in particles with a branch length between 30 and 45 nm, an inner shell edge-to-branch tip distance between 5 and 15 nm, and a gold shell thickness between 4 and 8 nm. Summed heat loss values for the different models increase rapidly from 1 to 4 nm in shell thickness, at which point they level off from 5 to 8 nm shell thickness. The model shown in FIG. 3C produces the highest internal heat loss values, with a branch length of 35 nm, a branch tip to inner shell edge distance of 5 nm, and a shell thickness of 5 nm. FIGS. 3D and 3E show the normalized electric field |E|/|Eo| for the entire particle model and only the interior water cavity. The area of greatest enhancement occurs in the ring-shaped region at the junction of the branch and shell, seen clearly in FIG. 3E, with a maximum |E|/|Eo| value of 234.6 V/m. FIG. 3F contains the interior water cavity's cross-section, illustrating significant enhancement propagating along the branch. In the |E|/|Eo| map of the models, the greatest internal heat loss values are generated with a branch tip to inner shell edge distance of 5, 10, and 15 nm (FIG. 3G). FIG. 3G highlights how a variety of morphologies can generate a substantial field enhancement at 785 nm. These particle models generate maximum |E|/|Eo| values of 234.6, 216.1, and 203.3 V/m, respectively.

Plasmonic-active gold-coated caged gold nanostars (C-GNS) are described herein. The gold-coated C-GNS include: one or more C-GNS having a hollow gold shell surrounding an essentially spherical core and a plurality of gold branches protruding out from the core, wherein a thickness of the gold shell is essentially uniform and tunable. In addition, the one or more C-GNS are essentially free of surfactant and have a tunable plasmon resonance in the near infrared (NIR). The gold-coated G-GNS can be produced using either a galvanic replacement or a galvanic replacement-free reaction as described herein. When produced using the galvanic replacement-free reaction described herein, the gold-coated G-GNS have a reduced amount of gold-silver alloy in the gold shell relative to C-GNS produced through a galvanic replacement reaction. Optionally, the gold-coated C-GNS include one or more compounds sealed within the hollow gold shell. The one or more compounds can be a dye or a drug.

The reduced amount of gold-silver alloy in the gold shell of the C-GNS described herein, relative to C-GNS produced through a galvanic replacement reaction, can be a reduction of gold-silver alloy of at least 75%.

The C-GNS described herein can have a length of the branches ranging from 10-50 nm, a distance from the inner edge of the shell to the tip of the branches ranging from 1-20 nm, and the thickness of the gold shell ranges from 1-15 nm.

The C-GNS with the described physical parameters can comprise a local electric field enhancement ([E]/[E0]) from the visible to the NIR spectral range, relative to a plasmonic-active gold nanostar of a similar diameter that lacks the hollow gold shell. The plasmonic-active C-GNS particles generate a greater local electric field enhancement ([E]/[E0]) in the visible/NIR spectral range relative to a plasmonic-active gold nanostar of a similar diameter that lacks the hollow gold shell.

The plasmonic-active C-GNS particles retain a greater degree of NIR absorption intensity relative to a plasmonic-active gold nanostar that lacks the hollow gold shell. The greater degree of NIR absorption intensity can be at least 20%.

A diameter of the C-GNS can be 300 nm, 200 nm, or 100 nm or less.

The C-GNS can comprise an attached bioreceptor including one or a combination of an antibody, a nanobody, an antigen, a peptide, an aptamer, a nucleic acid, an enzyme, a molecular sentinel (MS), or an inverse molecular sentinel (iMS). The bioreceptor can comprise an optical reporter.

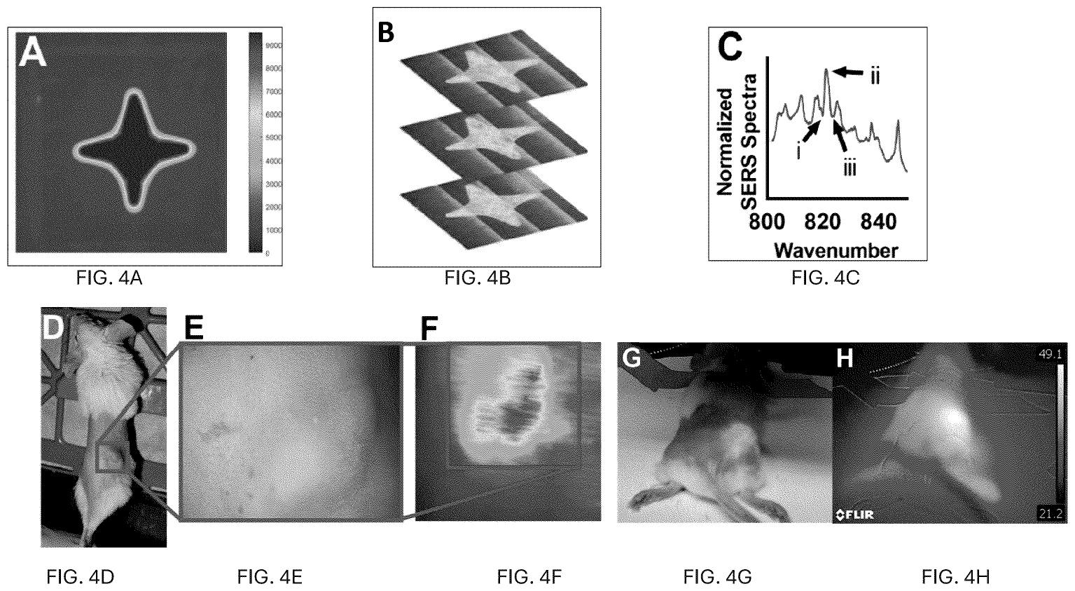

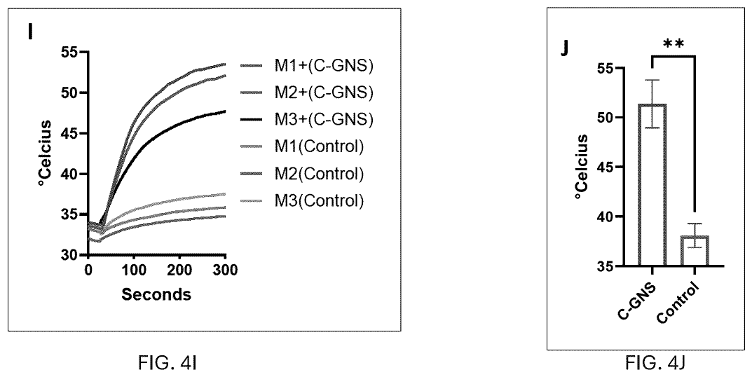

Gold-coated C-GNSs can be used as theranostic nanoparticles for photothermal therapy. FIG. 4A shows the total emission of the AU_2 formulation phantom C-GNS when excited with 785 nm light. Direct images of this phantom at 820, 822, and 824 nm wavelengths are shown in FIG. 4B in ascending order. The reference spectra of HITC dye in C-GNS is shown in FIG. 4C, where the labels I, ii, and iii correspond to the bottom, middle and top slices of FIG. 4B. While there is a significant signal due to the background fluorescence of the dye, there is a noticeable increase in the signal of the center slice in FIG. 4B.

FIGS. 4D-4J show the utility of gold-coated C-GNS for use in vivo for photothermal therapy. In the experiment, the gold-coated C-GNS are administered via retroorbital injection to mice with a bladder cancer flank tumor. FIG. 4D shows a mouse 24 hours after nanoparticle injection; the tumor is in the highlighted region. FIG. 4E is a bright field image of the tumor region through the lab-built microscope system, and FIG. 4F shows the hyperspectral image of the same region at 822 nm. The size of the C-GNS particles allows for passive accumulation within the tumor microenvironment. The high similarity between the bright field and hyperspectral images demonstrates this. The HITC dye encapsulated within the hollow region of the C-GNS enables the localization of the particles within the animal. The tumor region of each mouse is irradiated with light. The bright field and IR camera images of the photothermal treatment of a mouse that had received C-GNS are shown in FIGS. 4G and 4H. This marked temperature increase is caused by the local increase in NIR absorption generated by the particles. The temperature results of a hypodermic thermocouple placed into the center of the tumor are shown as a function of time for each mouse in FIG. 4I. The maximum values of each thermal camera measurement are shown in FIG. 4J. A sharp increase in temperature is seen in thermocouple data upon laser activation for all animals that received C-GNS. The average maximum temperature value seen for the group that received C-GNS was 51.4° C., whereas the average of the control group was 38.1° C. The unit used to evaluate clinical hyperthermia is cumulative effective minutes (CEM) at 43° C. over 90% of the tumor region. Irreversible tissue damage begins to occur at a value of 10 CEM4390. For example, if 90% of the tumor volume were 50° C. for 1 minute, the equivalent CEM4390 value would equal 128, far exceeding the threshold to induce cellular damage. These data demonstrate the potential of C-GNS particles for treating solid tumors via photothermal therapy.

The method of claim 14, wherein the optical reporter is selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, crystal violet, a fluorescence label, and absorbance label.

The C-GNSs described herein have intense SERS signals that can be tuned over a large spectral range. In addition, multiple dyes and/or drugs can be loaded into the C-GNS cavity structures (i.e., the hollow shells). The C-GNSs can serve as a stable internal and external standard under harsh in vivo and in vitro systems. The C-GNSs can be integrated with iMS nanosensors for in vitro and in vivo sensing. The multimodal C-GNS probe can be detected using SERS, Fluorescence, TPL, CT, or MRI. The C-GNSs can be integrated with magnetic particles and plasmonics coupling interferences system. The C-GNSs can be integrated with magnetic particles for nucleic target concentration, separation, hybridization with DNA probes for biosensing. The C-GNSs can be integrated with magnetic particles for protein (or small molecules) target concentration, separation, capture using antibodies (or aptamers), and biosensing. The C-GNSs can be integrated with magnetic particles for protein (or small molecules) target concentration, separation, capture using antibodies (or aptamers), and biosensing. The C-GNSs can be used for effective chemo or immunotherapy by local release of drug. The C-GNSs+iMS can be used for both detection and siRNA therapy.

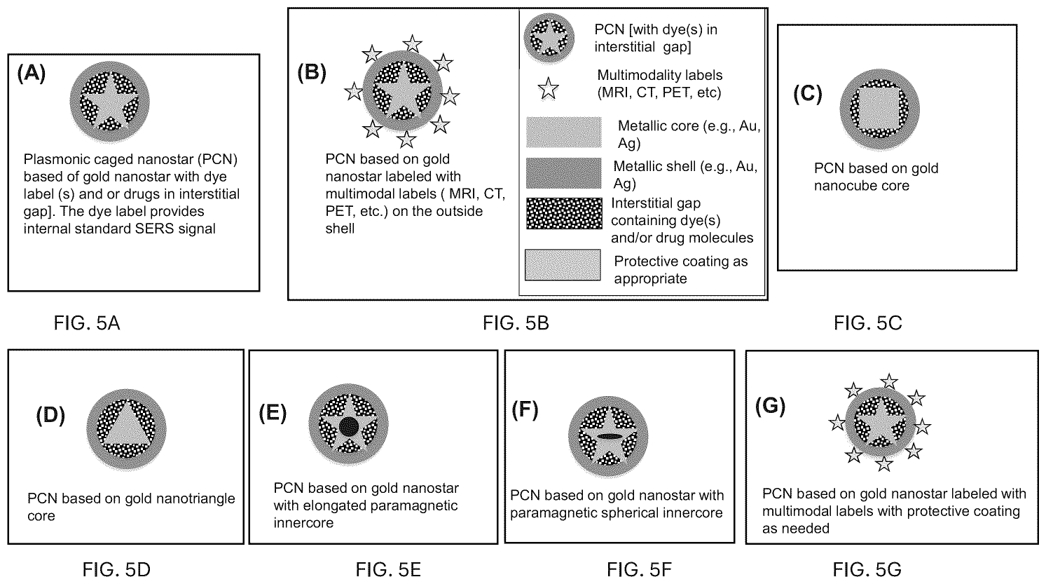

FIG. 5 shows the various embodiments of C-GNS probes that can be designed. FIG. 5A shows plasmonic caged nanostar (C-GNS) based on a gold nanostar with dye label(s) and/or drugs in the interstitial gap. The dye label provides internal standard SERS signal. FIG. 5B shows C-GNS based on a gold nanostar labeled with multimodal labels (MRI, CT, PET, etc.) on the outside shell. FIG. 5C shows C-GNS based on a gold nanocube core. FIG. 5D shows C-GNS based on a gold nanotriangle core. FIG. 5E shows C-GNS based on a gold nanostar with a paramagnetic spherical inner core. FIG. 5F shows C-GNS based on a gold nanostar with an elongated paramagnetic inner core. FIG. 5G shows C-GNS based on a gold nanostar labeled with multimodal labels with a protective coating as needed.

Bioreceptors are the key to specificity for targeting diseased cells, mutated genes, or specific biomarkers. The bioreceptors are responsible for binding the biotarget of interest to the drug system for therapy. The bioreceptors can take many forms and the different bioreceptors that have been used are as numerous as the different analytes that have been monitored using biosensors. However, bioreceptors can generally be classified into five different major categories. These categories include: 1) antibody/antigen, 2) enzymes, 3) nucleic acids/DNA, 4) cellular structures/cells and 5) biomimetic (aptamers, peptides, etc.).

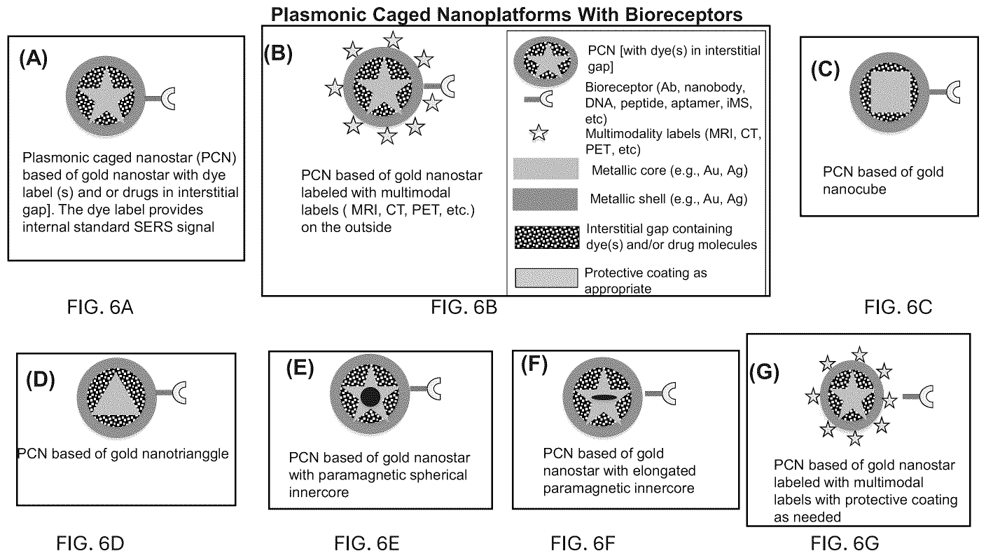

FIGS. 6A-6G show the various embodiments of C-GNS probes with attached bioreceptors (antibody, nanobody, DNA, peptides, aptamers, Mol Sentinel, iMS, etc.). The probes are similar to those in FIG. 5 but also have a bioreceptor, for example, for tumor targeting.

The operation of gene probes is based on the hybridization process. Hybridization involves the joining of a single strand of nucleic acid with a complementary probe sequence. Hybridization of a nucleic acid probe to DNA biotargets (e.g., gene sequences of a mutation, etc.) offers a very high degree of accuracy for identifying DNA sequences complementary to that of the probe. Nucleic acids strands tend to be paired to their complements in the corresponding double-stranded structure. Therefore, a single-stranded DNA molecule will seek out its complement in a complex mixture of DNA containing large numbers of other nucleic acid molecules. Hence, nucleic acid probe (i.e., gene probe) detection methods are very specific to DNA sequences. Factors affecting the hybridization or reassociation of two complementary DNA strands include temperature, contact time, salt concentration, and the degree of mismatch between the base pairs, and the length and concentration of the target and probe sequences.

A biologically active DNA probe can be immobilized onto the C-GNS described herein. Attached to the C-GNS, the gene probe is stabilized and, therefore, can be reused repetitively.

An antibody can be immobilized onto the C-GNS described herein. Antibodies are biological molecules that exhibit very specific binding capabilities for specific structures. This is very important due to the complex nature of most biological systems. An antibody is a complex biomolecule, made up of hundreds of individual amino acids arranged in a highly ordered sequence. For an immune response to be produced against a particular molecule, a certain molecular size and complexity are necessary: proteins with molecular weights greater then 5000 Da are generally immunogenic. The way in which an antigen and its antigen-specific antibody interact may be understood as analogous to a lock and key fit, by which specific geometrical configurations of a unique key enables it to open a lock. In the same way, an antigen-specific antibody “fits” its unique antigen in a highly specific manner. This unique property of antibodies is the key to their usefulness in immunosensors where only the specific analyte of interest, the antigen, fits into the antibody binding site.

An enzyme can be immobilized onto the C-GNS described herein. Enzymes are often chosen as bioreceptors based on their specific binding capabilities as well as their catalytic activity. In biocatalytic recognition mechanisms, the detection is amplified by a reaction catalyzed by macromolecules called biocatalysts. With the exception of a small group of catalytic ribonucleic acid molecules, all enzymes are proteins. Some enzymes require no chemical groups other than their amino acid residues for activity. Others require an additional chemical component called a cofactor, which may be either one or more inorganic ions, such as Fe²⁺, Mg²⁺, Mn²⁺, or Zn²⁺, or a more complex organic or metalloorganic molecule called a coenzyme. The catalytic activity provided by enzymes allows for much lower limits of detection than would be obtained with common binding techniques. The catalytic activity of enzymes depends upon the integrity of their native protein conformation. If an enzyme is denatured, dissociated into its subunits, or broken down into its component amino acids, its catalytic activity is destroyed. Enzyme-coupled receptors can also be used to modify the recognition mechanisms.

The immobilization of biomolecules such as, for example, drugs, proteins, enzymes, antibodies, DNA, etc. to a solid support can use a wide variety of methods published in the literature. Binding can be performed through covalent bonds usually takes advantage of reactive groups such as amine (—NH2) or sulfide (—SH) that naturally are present or can be incorporated into the biomolecule structure. Amines can react with carboxylic acid or ester moieties in high yield to form stable amide bonds. Thiols can participate in maleimide coupling, yielding stable dialkylsulfides. In one embodiment, receptor biomolecules are linked to gold (or silver-coated) C-GNSs. The majority of immobilization schemes involving Au (Ag) surfaces utilize a prior derivatization of the surface with alkylthiols, forming stable linkages. Alkylthiols readily form self-assembled monolayers (SAM) onto silver surfaces in micromolar concentrations. The terminus of the alkylthiol chain can be used to bind biomolecules or can be easily modified to do so. The length of the alkylthiol chain has been found to be an important parameter, keeping the biomolecules away from the surface. Furthermore, to avoid direct, non-specific DNA adsorption onto the surface, alkylthiols have been used to block further access to the surface, allowing only covalent immobilization through the linker [Steel, A. B.; Herne, T. M.; Tarlov, M. J. Anal. Chem. 1998, 70, 4670-7; Herne, T. M.; Tarlov, M. J. J. Am. Chem. Soc. 1997, 119, 8916-20].

Silver surfaces have been found to exhibit controlled self-assembly kinetics when exposed to dilute ethanolic solutions of alkylthiols. The tilt angle formed between the surface and the hydrocarbon tail ranges from 0 to 15□. There is also a larger thiol packing density on silver, when compared to gold [Burges, J. D.; Hawkridge, F. M. Langmuir 1997, 13, 3781-6]. After SAM formation on gold/silver nanoparticles, alkylthiols can be covalently coupled to biomolecules. The majority of synthetic techniques for the covalent immobilization of biomolecules utilize free amine groups of a polypeptide (enzymes, antibodies, antigens, etc.) or of amino-labeled DNA strands, to react with a carboxylic acid moiety forming amide bonds. As a general rule, a more active intermediate (labile ester) is first formed with the carboxylic acid moiety and in a later stage reacted with the free amine, increasing the coupling yield. Successful coupling procedures include the following listed below.

Binding Procedure Using N-hydroxysuccinimide (NHS) and its derivatives. The coupling approach involves the esterification under mild conditions of a carboxylic acid with a labile group, an N-hydroxysuccinimide (NHS) derivative, and further reaction with free amine groups in a polypeptide (enzymes, antibodies, antigens, etc.) or amine-labeled DNA, producing a stable amide [Boncheva, M.; Scheibler, L.; Lincoln, P.; Vogel, H.; Akerman, B. Langmuir 1999, 15, 4317-20]. NHS reacts almost exclusively with primary amine groups. Covalent immobilization can be achieved in as little as 30 minutes. Since H2O competes with —NH2 in reactions involving these very labile esters, it is important to consider the hydrolysis kinetics of the available esters used in this type of coupling. The derivative of NHS used in FIG. 1, O—(N-succinimidyl)-N, N,N′,N′-tetramethyluronium tetrafluoroborate, increases the coupling yield by utilizing a leaving group that is converted to urea during the carboxylic acid activation, hence favorably increasing the negative enthalpy of the reaction.

Binding Procedure Using Maleimide. Maleimide can be used to immobilize biomolecules through available-SH moieties. Coupling schemes with maleimide have been proven useful for the site-specific immobilization of antibodies, Fab fragments, peptides, and SH-modified DNA strands. Sample preparation for the maleimide coupling of a protein involves the simple reduction of disulfide bonds between two cysteine residues with a mild reducing agent, such as dithiothreitol, 2-mercaptoethanol or tris(2-carboxyethyl) phosphine hydrochloride. However, disulfide reduction will usually lead to the protein losing its natural conformation and might impair enzymatic activity or antibody recognition. The modification of primary amine groups with 2-iminothiolane hydrochloride (Traut's reagent) to introduce sulfydryl groups is an alternative for biomolecules lacking them. Free sulfhydryls are immobilized to the maleimide surface by an addition reaction to unsaturated carbon-carbon bonds.

Binding Procedure Using Carbodiimide. Surfaces modified with mercaptoalkyldiols can be activated with 1,l′-carbonyldiimidazole (CDI) to form a carbonylimidazole intermediate. A biomolecule with an available amine group displaces the imidazole to form a carbamate linkage to the alkylthiol tethered to the surface [Potyrailo, R. A., et al., 1998].

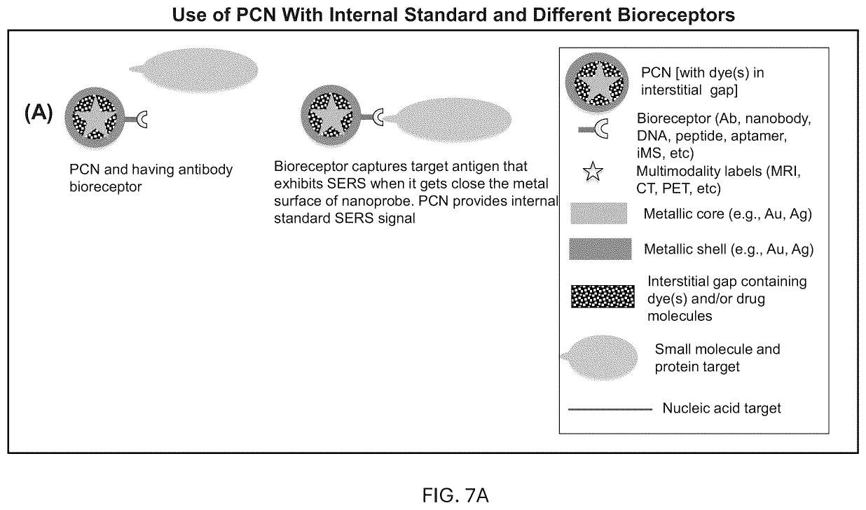

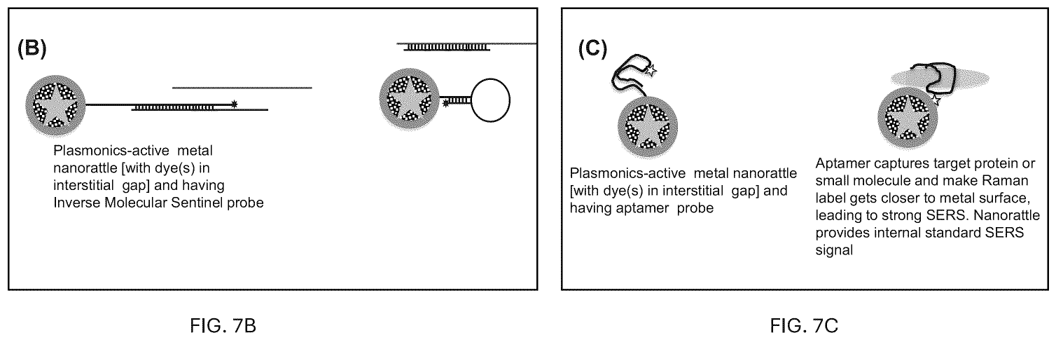

FIGS. 7A-7C illustrate the different uses of the C-GNS probes in various bioassays. FIG. 7A shows a C-GNS having an antibody (or nanobody) bioreceptor which captures a target antigen that exhibits SERS when the antigen gets close to the metal surface of the nanoprobe. The C-GNS provides an internal standard SERS signal. FIG. 7B shows a C-GNS having an Inverse Molecular Sentinel (iMS) probe which when a target nucleic acid (DNA, mRNA, miRNA) hybridizes to the probe it displaces the capture molecule and the iMS probe forms a stem loop structure leading to a SERs signal. The C-GNS provides an internal standard SERS signal. FIG. 7C shows a C-GNS having an aptamer probe which captures target protein or small molecule which causes the Raman label to locate closer to the metal surface, leading to strong SERS. The C-GNS provides internal standard SERS signal.

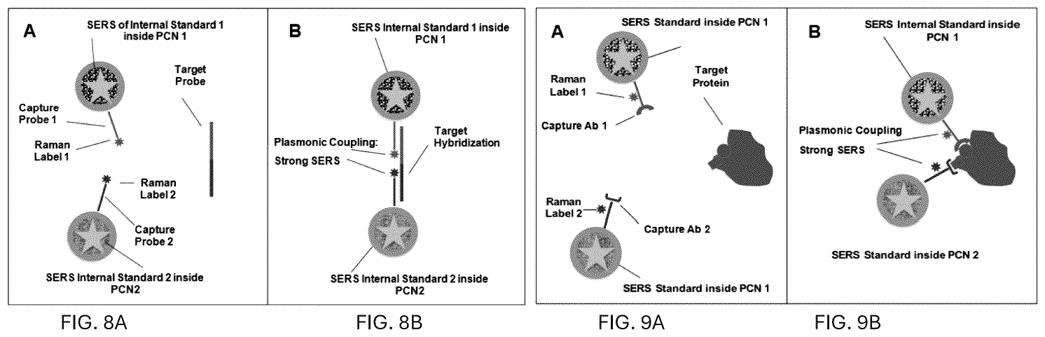

FIGS. 8A-8B illustrate that the C-GNSs can be used for detecting nucleic acid targets. FIG. 8A shows two plasmonics-active C-GNS nanoparticles (NPs), each having a SERS dye embedded inside the interstitial core-shell gap and having a probe DNA sequence. The C-GNS 1 probe (which has a SERS dye 1 embedded inside the interstitial core-shell gap) has a DNA 1 sequence (RED color) identical to the half target sequence of interest and has a Raman label bound at the end of the probe. The C-GNS 2 probe (which has a SERS dye 2 embedded inside the interstitial core-shell gap) has a DNA sequence (BLUE color) identical to the other half target sequence of interest and has a Raman label bound at the end of the probe.

When these two types of C-GNSs are mixed with the target sequence, they hybridize to the target probe in such a way that the SERS labels are in the middle (FIG. 8B). As a result, the two Raman labels are “trapped” between the two metal nanoparticles. Due to the interparticle plasmonics coupling described above, upon excitation (e.g., using a laser or other appropriate energy sources) of the label molecules, the electromagnetic enhancement of the Raman signal is very intense, leading to extremely strong SERS signals of the two Raman labels. The increase of the SERS signal intensities of the two Raman labels can be used as a parameter for monitoring and quantitatively detecting the target DNA/RNA in the assay. At the same time the SERS signals of the dye labels inside the C-GNS 1 and C-GNS 2 can be used as the internal standards. In some embodiments, the C-GNS 1 and C-GNS 2 have the same dye.

FIG. 9 illustrates that the C-GNSs described herein can be used to detect protein targets. FIG. 9A shows two plasmonics-active C-GNSs, each having a bioreceptor (e.g., capture antibody target to specific protein or a cancer cell). The 1^{st} C-GNS 1 probe (which has a SERS dye embedded inside the interstitial core-shell gap) has a Raman label attached to a bioreceptor targeted to a specific region (or cell surface receptor) of a cancer cell (RED color). The second C-GNS 2 probe (which has a SERS dye embedded inside the interstitial core-shell gap) has another Raman label bound to a bioreceptor targeted to another specific region of the protein (or another cell surface receptor of the cancer cell) (BLUE color).

When these two types of nanoprobes are mixed with the target proteins (or cancer cell, respectively), they bind to the target in such a way that the two Raman labels are in the middle of the two C-GNSs (FIG. 9B). As a result, the two Raman labels are “trapped” between the two metal nanoparticles. Due to the interparticle plasmonics coupling described above, upon excitation (e.g., using a laser or other appropriate energy sources) of the label molecules, the electromagnetic enhancement of the Raman signal is very intense, leading to extremely strong SERS signals of the two Raman label. The increase of the SERS signal intensities of the two Raman labels can be used as a parameter for monitoring and quantitatively detecting the protein target in the assay. At the same time the SERS signal of the dye inside the C-GNSs can be used as the internal standard.

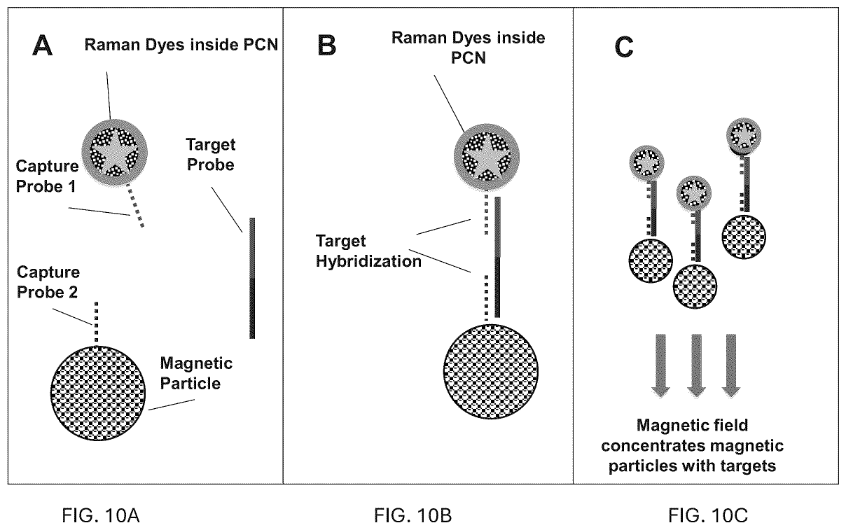

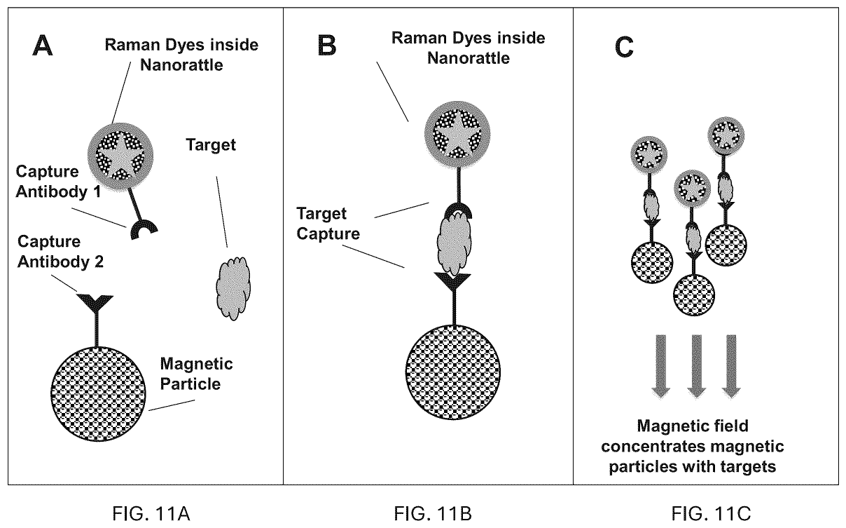

FIGS. 10A-10B and 11A-11B illustrate that the C-GNS probes described herein can be attached to magnetic beads to concentrate the samples with a magnetic field for detection of nucleic acid target and protein target, respectively.

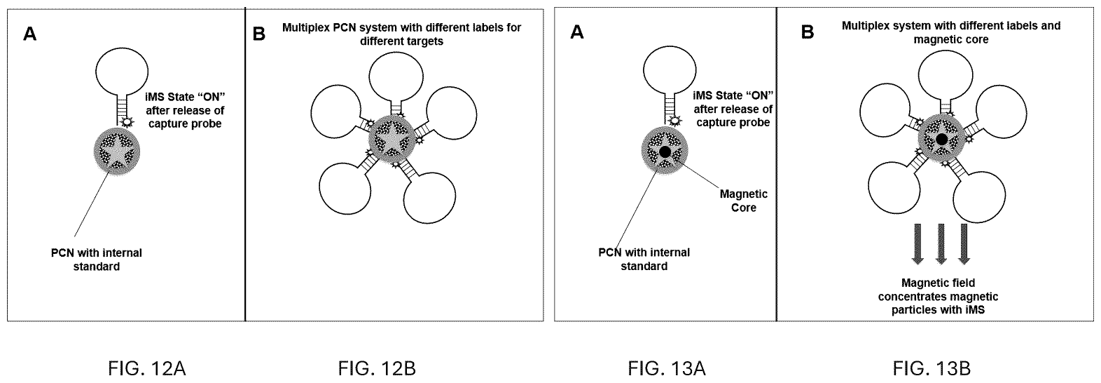

FIGS. 12A-12B show an embodiment of a multiplex C-GNS system using a C-GNS having an attached iMS probe with different labels for detection of different nucleic acid targets.

FIGS. 13A-13B show an embodiment of a multiplex C-GNS system using a C-GNS having a magnetic core and an attached iMS probe with different labels for detection of different nucleic acid targets. In this embodiment the magnetic C-GNS probes can be concentrated with a magnetic field.

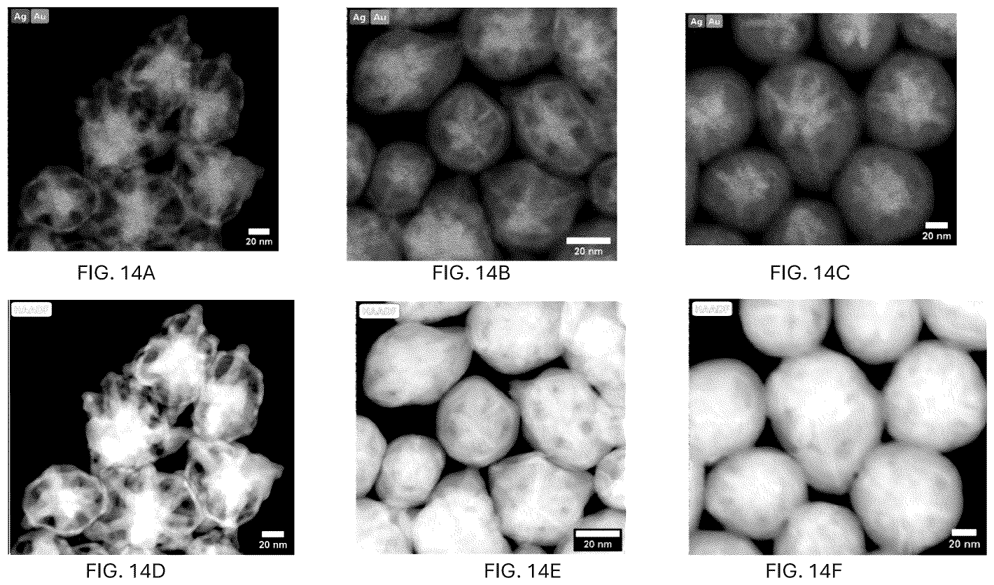

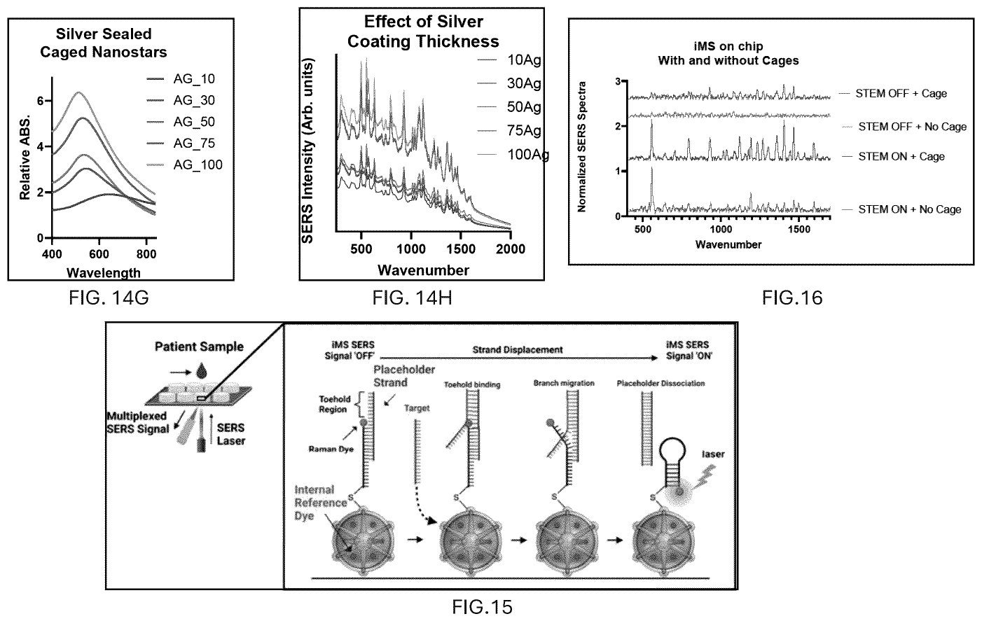

In addition to coating the C-GNSs with gold to seal the cargo loaded within the hollow shell, the C-GNSs can alternatively be sealed with a coating of silver. FIG. 14 shows how the C-GNSs can be sealed with variable amounts of silver, effecting the optical absorption and plasmonic enhancement of the particles thereby forming the silver-coated C-GNSs described herein. FIGS. 14A-14C contain STEM-EDS images of C-GNS particles coated with an increasing thickness of silver. The subsequent change in the particle absorption spectra is shown in FIGS. 14D-14F. The SERS spectra as a function of silver-coating thickness is shown in FIGS. 14G and 14H for silver-coated C-GNS particles loaded with HITC dye. The SERS signal increases with silver shell thickness.

Plasmonic-active silver-coated caged gold nanostars (C-GNS) are provided. The silver-coated C-GNS include: one or more C-GNS having a hollow gold shell coated with silver surrounding an essentially spherical core and a plurality of gold branches protruding out from the core, wherein a thickness of the silver-coated gold shell is essentially uniform and tunable. In addition, the one or more silver-coated C-GNS are essentially free of surfactant and have a tunable plasmon resonance in the near infrared (NIR). Optionally, the silver-coated C-GNS include one or more compounds sealed within the hollow silver-coated gold shell. The one or more compounds can be a dye or a drug.

FIG. 15 shows a conceptual drawing of a C-GNS functionalized substrate integrated into device-based biomarker sensing applications. In this embodiment, the patient sample can be added directly to the chip and, after biomarker recognition, a SERS laser interrogates the sample and the resulting multiplexed SERS spectra is obtained. The inset in FIG. 15 depicts the C-GNS particles having an attached iMS sensing probes used for the amplification-free multiplexed detection of miRNA biomarkers. In addition, the C-GNS iMS sensing probes have an internal dye within the hollow core which serves as a reference signal.

FIG. 16 shows an example of the differences between the spectra of bimetallic nanostars that lack a cage structure (No Cage) in both the “ON” and “OFF” positions and bimetallic C-GNS particles that have a cage (Cages) in the iMS assay. The SERS signal of the STEM loop in the off position on the Cage (C-GNS) substrate is from the dye encased within the particle. This signal can be used to standardize the number of particles per area and, therefore, probe per area. With this internal standard, quantitative amplification-free multiplexed detection of cancer biomarkers can be performed.

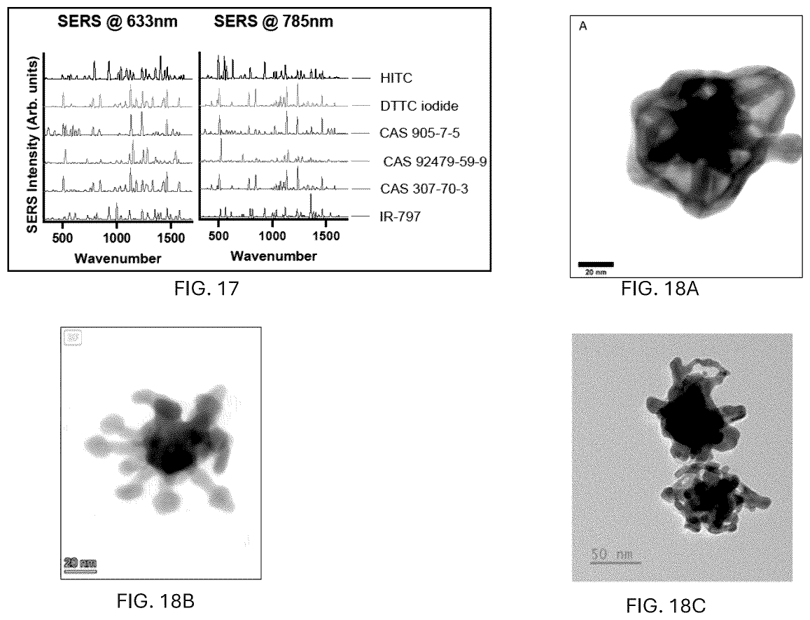

FIG. 17 shows the SERS spectra of C-GNS particles filled with different molecules, taken at two different SERS wavelengths commonly used in sensing applications.

The Tunable plasmonic properties of the C-GNSs combined with their simple loading procedure can quickly lead to a robust library of multiplexed SERS probes, with their surface free for biosensor functionalization. FIGS. 18A-18C show TEM images of caged nanostars filled with dye molecules. More specifically, FIG. 18A shows a gold-sealed C-GNS particle filled in this example with dye molecules. The gold-sealed C-GNS particles act as leakproof transporters that can carry and concentrate chemotherapeutics via the EPR effect into the tumor microenvironment. FIG. 18B demonstrates that via selective chemical etching, the shell of the C-GNS can be removed. This is a proof-of-concept demonstration for the triggered, local opening of the cage to release the contents inside. FIG. 18C shows selectively opened C-GNS particles where the selective opening was performed with a pulsed laser. This is an example of a method that can be used in vivo.

A method is provided for delivery of a compound to a local environment, comprising: administering to a subject a plasmonic-active caged gold nanostar as described herein having one or more compounds loaded within the hollow gold shell, wherein the plasmonic-active caged gold nanostar particles travel to the local environment, and applying laser excitation to the local environment to open the hollow gold shell of the nanostar particles, thereby delivering the compound to the local environment. The local environment can be a local tumor environment and the particles can travel to the local tumor environment within the subject through an enhanced permeability effect and retention effect (EPR). In another aspect, the C-GNS can comprise a bioreceptor that targets the C-GNS to the local environment. The one or more compounds can comprise one or both of an optical reporter and a chemotherapeutic. The method can further include detecting the plasmonic-active caged gold nanostars in the local environment through detection of the optical reporter. The administering can be via oral delivery, intra-arterial injection, or intravenous injection.

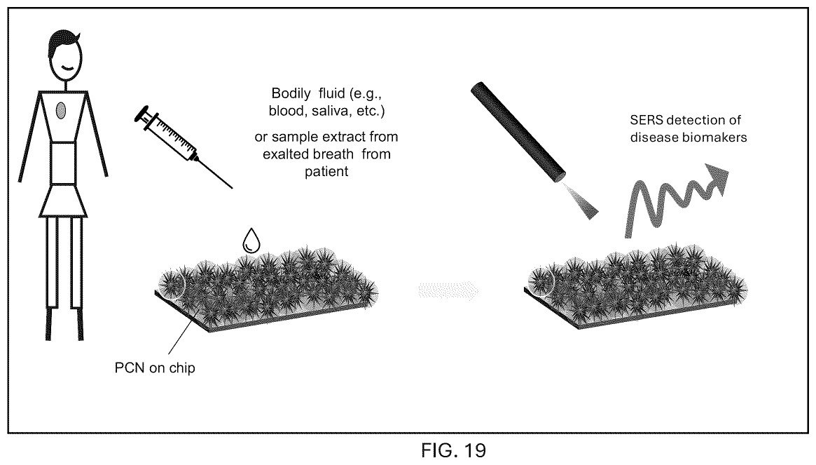

FIG. 19 illustrates the basic operating principle of the in vitro diagnostics modality using C-GNS probes. In this embodiment, the C-GNS is on a chip and a bodily fluid sample such as, but not limited to, blood, saliva, or exhaled breadth is extracted from the patient and contacted with the C-GNS on the chip. The target of biomarker of interest is then detected on the chip using SERS.

A method is provided for in vitro detection of a target of interest, comprising: contacting one or more plasmonic-active caged gold nanostar particles as described herein with a sample, wherein the one or more plasmonic-active caged gold nanostar particles further comprises a bioreceptor for one or more of a cell, a nucleic acid, or a protein target of interest and, optionally, an optical reporter; and detecting an optical signal from one or both of the optical reporter and the one or more compounds loaded within the hollow gold shell. In the method, the bioreceptor can comprise a nucleotide sequence, an aptamer, an antibody, an enzyme, or a cell-based receptor. The optical signal can be a Raman signal or a surface-enhanced Raman scattering (SERS) signal.

The bioreceptor can comprise: (i) a stem-loop nucleic acid probe comprising a sequence that forms a stem loop, a first end attached to the one or more plasmonic-active caged gold nanostar particles, and a second end labeled with the optical reporter, and (ii) an unlabeled capture placeholder nucleic acid strand comprising a first region complementary to a sequence in a nucleic acid target of interest and a second region complementary to the sequence that forms a stem-loop or a portion thereof, wherein the second region is shorter than and overlapping the first region, wherein in the presence of the nucleic acid target the placeholder nucleic acid strand binds to the sequence in the nucleic acid target of interest and the stem loop closes, thereby inducing the optical signal for detection. The nucleic acid target of interest can be a microRNA, a small noncoding RNA, an mRNA, or a DNA sequence.

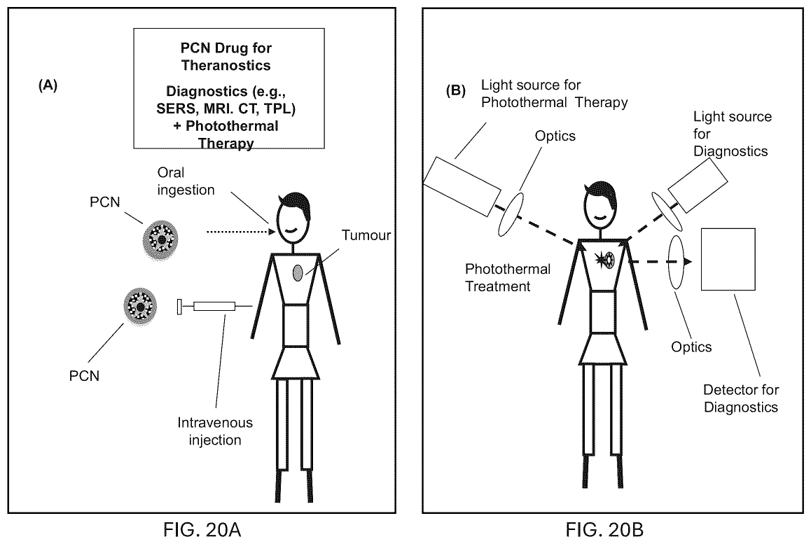

FIG. 20 illustrates the basic operating principle of the in vivo diagnostic and therapy modality using C-GNS probes. The C-GNS nanoprobe particles are given to a person by injection or oral delivery into a person using various methodologies. In one embodiment, the C-GNS probes having magnetic cores can be moved to and concentrated in an area suitable for detection. Several diagnostics systems are possible, depending on the degree of miniaturization. For example, detection of the target can be through use of a portable Raman diagnostic system having excitation light source and an optical detector. An alternative diagnostic system consists of a pocket-sized (or palm-sized) battery-operated Raman diagnostics system that is linked to the ‘smart mole’ by fiberoptics excitation and detection. The pocket-sized system can be operated remotely by an iPhone or similar device.

A method is provided for in vivo imaging, comprising: administering to a subject one or more C-GNSs as described herein, wherein one or more compounds loaded within the hollow gold shell comprises an optical reporter, and detecting an optical signal from the optical reporter. In the method, the one or more C-GNSs can include a bioreceptor to target the C-GNSs to a cell, protein, or nucleic acid of interest, the bioreceptor optionally comprising a second optical reporter, and the method optionally further comprising detecting a second optical signal from the second optical reporter when the bioreceptor is in the presence of the target of interest.

The bioreceptor can comprise a nucleotide sequence, an aptamer, an antibody, an enzyme, or a cell-based receptor. In one aspect, the bioreceptor can comprise: (i) a stem-loop nucleic acid probe comprising a sequence that forms a stem loop, a first end attached to the one or more plasmonic-active caged gold nanostar particles, and a second end labeled with the second optical reporter, and (ii) an unlabeled capture placeholder nucleic acid strand comprising a first region complementary to a sequence in the nucleic acid target of interest and a second region complementary to the sequence that forms a stem-loop or a portion thereof, wherein the second region is shorter than and overlapping the first region, wherein in the presence of the nucleic acid target the placeholder nucleic acid strand binds to the sequence in the nucleic acid target of interest and the stem loop closes, thereby inducing the second optical signal for detection. The nucleic acid target of interest can comprise a microRNA, a small noncoding RNA, an mRNA, or a DNA sequence.

A method is provided for in vivo photothermal therapy. The method comprises: administering to a subject one or more C-GNS as described herein, wherein the one or more C-GNSs travel to the local environment of interest; and irradiating the local environment of interest to generate an increase in NIR absorption by the particles, thereby increasing the temperature of the local environment of interest. The C-GNS can include one or an optical reporter loaded within the hollow gold shell. The local environment of interest can be a local tumor environment, and the one or more C-GNSs can travel to the local tumor environment through an enhanced permeability and retention effect (EPR). Alternatively, the one or more C-GNSs can include a bioreceptor to target the C-GNSs to a cell or protein of interest, the bioreceptor optionally comprising a second optical reporter, and the method optionally further comprising detecting a second optical signal from the second optical reporter when the bioreceptor is in the presence of the target of interest.

The optical signal can be a Raman signal or surface-enhanced Raman scattering (SERS) signal. The optical reporter can be selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, crystal violet, a fluorescence label, and absorbance label.

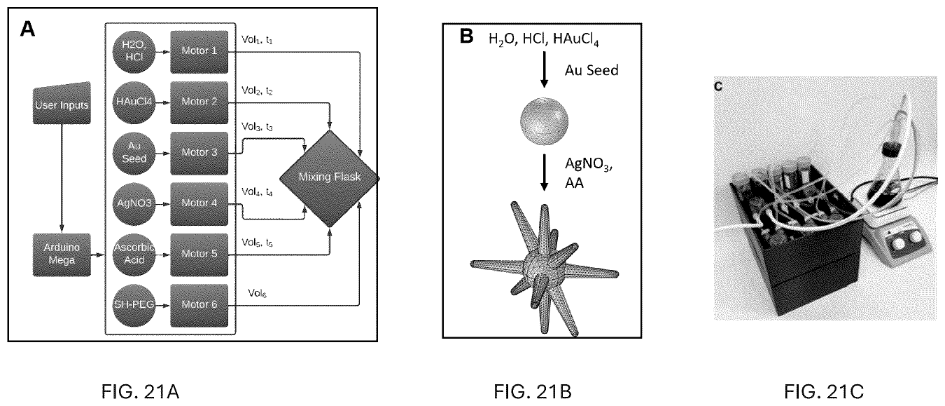

The inherent challenge of surfactant-free high-aspect ratio gold nanostar-based nanoparticles (i.e, GNS, BNS), and C-GNS is that their morphology is highly sensitive to variations in synthetic conditions. The surfactant-free procedure for generating these nanoparticles described herein requires the rapid and precisely timed addition of several reagents in immediate succession (see Example 2 and FIGS. 21-26). Varying the timing of any single reagent by only a handful of seconds can cause differences between batches of gold nanostar-based nanoparticles. Also, the gold nanostar-based nanoparticle reactions are sensitive to different local concentrations throughout the solution mixture when each reagent is added. This limits the volume of each nanoparticle batch, often requiring multiple batches for a single experiment.

In one embodiment, an automated plasmonic nanoparticle synthesis (AC-GNS) platform for improved nanoparticle fabrication and optimization is provided. The AC-GNS system can be a general manufacturing platform for large-scale, reproducible, optimized fabrication of gold nanostar, bimetallic nanostar, and caged nanostar systems as well as many other nanoparticle platforms.

Gold nanostar-based nanoparticles have gained much attention for their highly tunable optical properties. By varying nanoparticle parameters such as metallic composition, particle diameter, branch number, and branch aspect ratio, the localized surface resonance peak of these particles can be tuned between 500 nm and 1100 nm. The surfactant-free and, therefore, biocompatible GNS synthesis enables use of GNS in in vitro chemical and biomolecule sensing applications and their deployment in a wide range of cancer and other disease theragnostics. The surfactant-free C-GNS particles formed using the methods and devices described herein have dramatically improved in vitro sensing capabilities. In addition, the automated methods described herein below allow for GNS synthesis with greatly reduced batch-to-batch differences. Serial synthesis describes the process by which the necessary reagents for GNS-based nanoparticle synthesis are added sequentially with predetermined timing steps to a single mixing container. The schematic diagram shown in FIG. 21A outlines the active components of the GNS-based nanoparticle synthesis device. A user can make input into a microcontroller, which in turn controls a total of 6 motors that are used to dispense different reagents necessary for GNS-based nanoparticle synthesis. All volumes and timing between reagent injections are assignable for each run, allowing for the precise control of synthetic conditions. By adjusting these parameters, the optical properties of the GNS-based nanoparticle can be tuned to fit specific application needs.

FIG. 21B illustrates the synthetic steps that result in the formation of GNS nanoparticles. For a single batch of GNS, gold ion solution, followed by gold nanospheres, silver nitrate, and ascorbic acid is quickly added to a solution in a rapidly mixing flask to trigger branch formation on the surface of the gold nanospheres, resulting in the GNS morphology. The photograph in FIG. 21C shows the GNS synthesis device immediately after producing a batch of nanoparticles. All controlling circuitry is housed in the lower section of the GNS synthesis device housing. All peristaltic pumps were chosen for incorporation into the device to ensure no contamination from the pump head. For all 6 motors, one end of the tubing is placed into the reagent reservoir, and the other is fitted with a pipette tip and inserted into the injection head, which is fitted to the neck of the flask. This injection head ensures no contact between individual injection channels and all injection channels are oriented vertically directly at the mixing solution below.

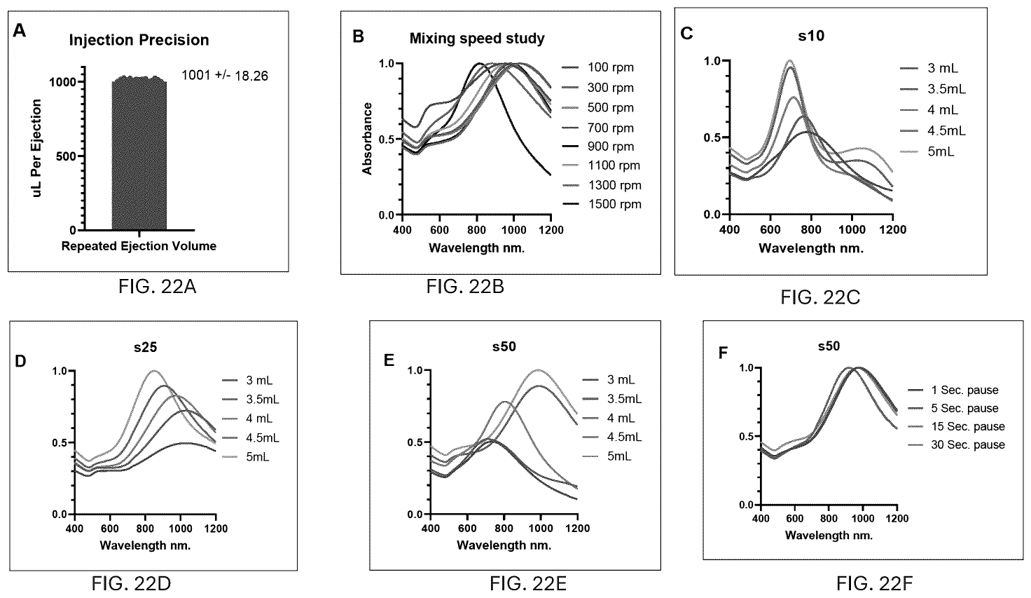

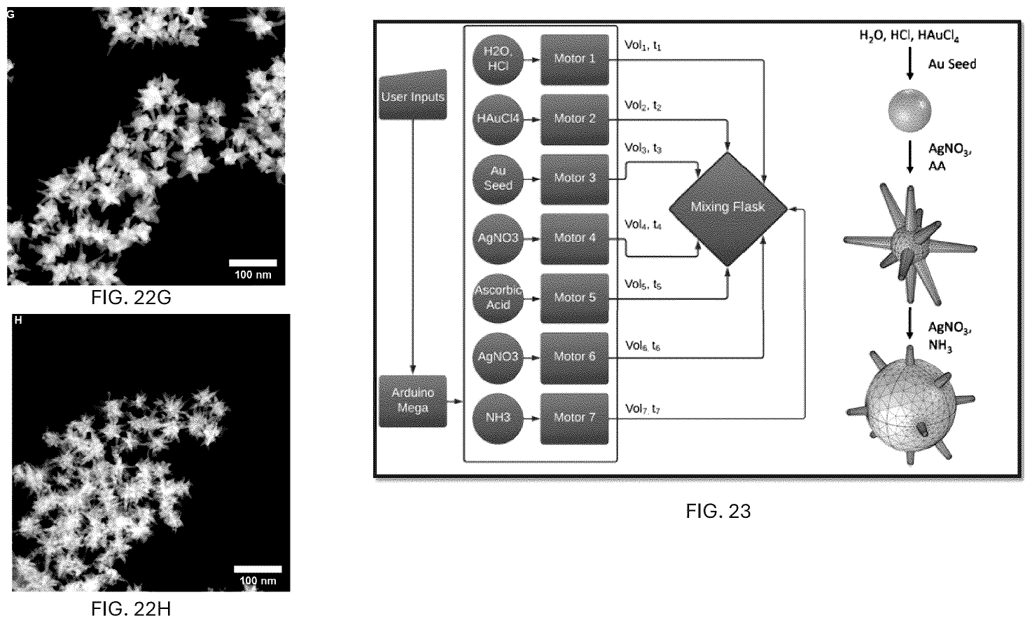

In this device, several variables can significantly impact the final morphology of GNS particles. These variables include the reaction volume, the quantities of all five reagents necessary to achieve stable GNS particles, the timing between all injection steps, the solution's mixing speed, and the injection volume variability. FIG. 22A shows the volumes of 30 injections in a row, averaging 1001 uL with a standard deviation of 18.26 uL, or 1.8%. The absorption spectra of all GNS solutions produced as a result of mixing speed study are shown in FIG. 22B. At the lowest speed, the absorption peak is the broadest, indicating the formation of highly heterogeneous GNS in solution. As the mixing speed increases from 300 to 1300 rpm, the absorbance spectra sharpen, and the peak absorption value blue shifts from 1028 nm to 882 nm. At the highest mixing speed, 1500 rpm, the absorbance spectra dramatically narrow, and the peak absorption value further blue shifts to 814 nm. This noticeable peak narrowing indicates an increase in the uniformity of GNS particles within the solution relative to other prepared batches. Plasmonic nanoparticles are generally used in applications like photothermal therapy or SERS sensing that use a single input/excitation wavelength. Therefore, if there is a large degree of homogencity between nanoparticles in the solution, the solution will interact more strongly with the input light than a solution containing more heterogeneous nanoparticles.

An automated method is provided for producing plasmonic-active gold nanostars. The method comprises adding to a single reaction vessel with rapid mixing, predetermined timing, and in sequential order the following reagents: (i) an acidic water solution, (ii) a gold ion solution, (iii) a plurality of gold nanospheres, (iv) a silver ion solution, and (v) an ascorbic acid solution, to trigger branch formation on a surface of the gold nanospheres, thereby forming plasmonic-active gold nanostars. The method can further comprise adding a thiolated polyethylene glycol solution to the formed plasmonic-active gold nanostars.

In the automated method, adding the reagents to the reaction vessel can be performed with a standard deviation in the amount of each reagent added of 5% or less, 4% or less, 3% or less, 2% or less, or 1.8% or less.

In the automated method, a mixing speed of the solution in the reaction vessel can be 1200 rpm or more, 1300 rpm or more, 1400 rpm or more, or 1500 rpm or more.

The absorption spectra for three different base GNS morphologies produced according to the automated method “s10”, “s25”, and “s50”, where the number refers to the final micromolar concentration of silver nitrate, are shown in FIGS. 22 C-E, respectively. For each of the base morphologies, the amount of gold chloride solution added in the method ranged between 3 mL and 5 mL in steps of 0.5 mL. Clear trends appear for all base GNS morphologies. Beginning with s10, as the amount of gold chloride in the solution is increased, the absorbance of the solution increases, the main peak value blue shifts from 782 nm to 696 nm, and the peak narrows. Also, a secondary peak at 1046 nm rises notably when 4.5 mL and 5 mL of gold chloride are added to the solution. A similar trend is seen in the s25 base morphology as well, as the amount of gold chloride increases, the peak absorption value increases, and blue shifts from 1030 nm to 850 nm while the peak is also narrowing. In the s50 base morphology, the peak absorbance value increases as more gold chloride is added to the solution, however, the value red shifts from 730 nm to 988 nm, while the peak is narrowing. By altering the base GNS morphology with the amount of silver introduced into the solution and the amount of gold chloride added, which controls the GNS branch formation, the optical therapeutic window can be effectively spanned.

FIG. 22F shows the average absorption spectrum of ten identical batches of S50 GNS particles prepared using the automated method. The average maximum absorbance value of all ten batches was 983.8 nm with a standard deviation of 9.1 nm. All injection amounts are held constant to investigate the effects of the specific timing of reagent addition. In contrast, the timing between the addition of silver nitrate and ascorbic acid is varied between 1 second, 5 seconds, 15 seconds, and 30 seconds, and all batches are prepared in triplicate. The absorbance spectra in FIG. 22F show a high degree of similarity between the batches with injection pauses of 1, 5, and 15 seconds. The batches with a pause of 30 seconds are blue shifted in comparison. Regardless of the duration of the reagent injection pause, there was almost no variation in the absorbance spectra of replicate batches. FIGS. 22G and 22H are HAADF STEM images of s10 particles prepared with 5 mL of gold chloride and s50 particles prepared with 5 mL of gold chloride, respectively, and confirm that the noticeable change in particle morphology is the cause of the changes in absorbance spectrum seen in FIGS. 22C-22E.

In one embodiment, the average maximum absorbance value of the plasmonic-active caged gold nanostars (C-GNS) has a standard deviation of 20 nm or less.

FIG. 23 shows that with one additional modular motor unit, the GNS synthesis device of FIG. 21A can be converted into an automatic synthesis machine for bimetallic nanostars. For BNS, the underlying chemistry shares many similarities with GNS synthesis, however, instead of adding SH-PEG5000 as the final step of the reaction, a second addition of silver nitrate occurs, followed by a dilute ammonium hydroxide solution. These differences cause a layer of silver to form on the core of the GNS particle and grow outwards to the nanoparticle branch tips. These BNS particles are highly tunable and can be used to provide signal enhancement for SERS biosensing applications. The method can further comprise adding a second addition of the silver nitrate solution to the formed plasmonic-active gold nanostars, followed by adding an ammonium hydroxide solution to cause a layer of silver to form on a core of the plasmonic-active gold nanostars and grow outwards to a tip of the branches, thereby forming plasmonic-active bimetallic nanostars.

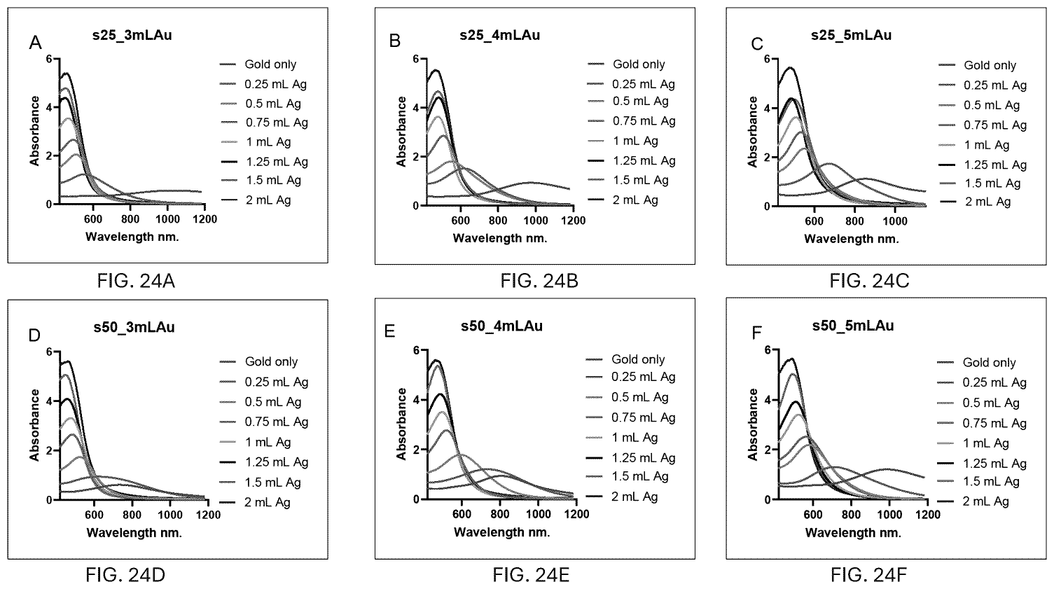

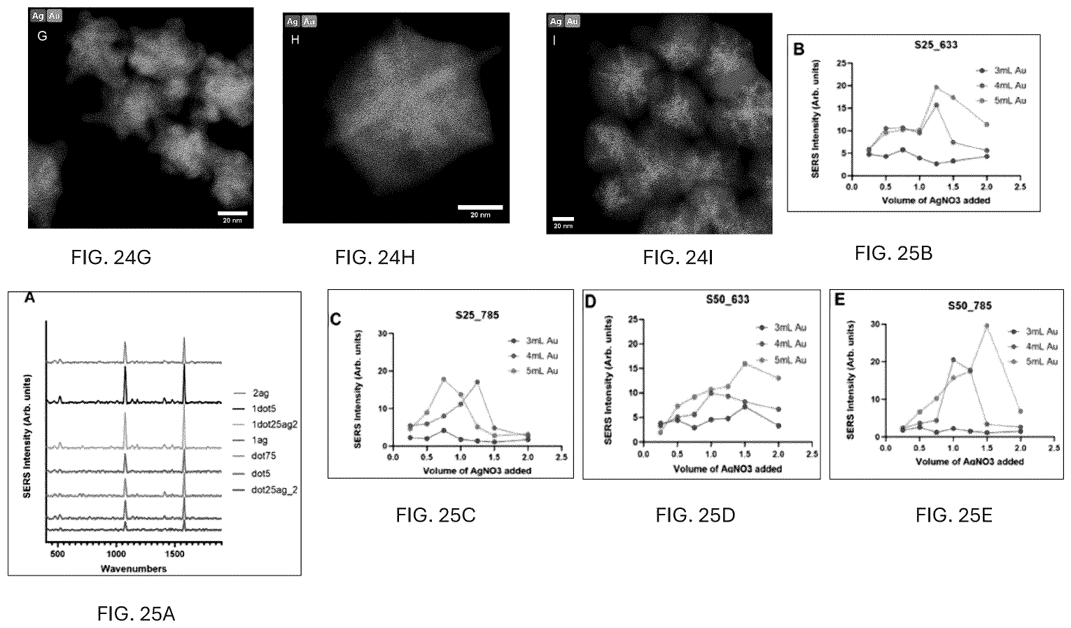

FIGS. 24A-24F show the absorbance spectra of the different base bimetallic GNS morphologies as the amount of silver added increases. FIGS. 24G-24I are STEM-EDS images 5 of S25_5 mL_dot25Ag particles, S25_5 mL_1dot25Ag particles, and S25_5 mL_2Ag particles, respectively, showing the changes in nanoparticle morphology as varying amounts of silver were reduced onto the surface of the GNS.

The performance of these BNS can be assessed by combination with a p-mercaptobenzoic acid (p-MBA) solution and subsequent SERS spectra at 633 nm and 785 nm. The automated plasmonic nanoparticle synthesis platform enables reproducible fine-tuning of nanoparticle morphology to generate the greatest degree of signal enhancement. FIG. 25A shows the SERS spectra of S25_5 mL particles with a silver addition amount ranging from 0.25 mL to 2 mL when labeled with p-MBA. FIGS. 25B-25E are graphs showing the peak height at 1586 cm⁻¹ for all synthesized formulations of BNS particles.

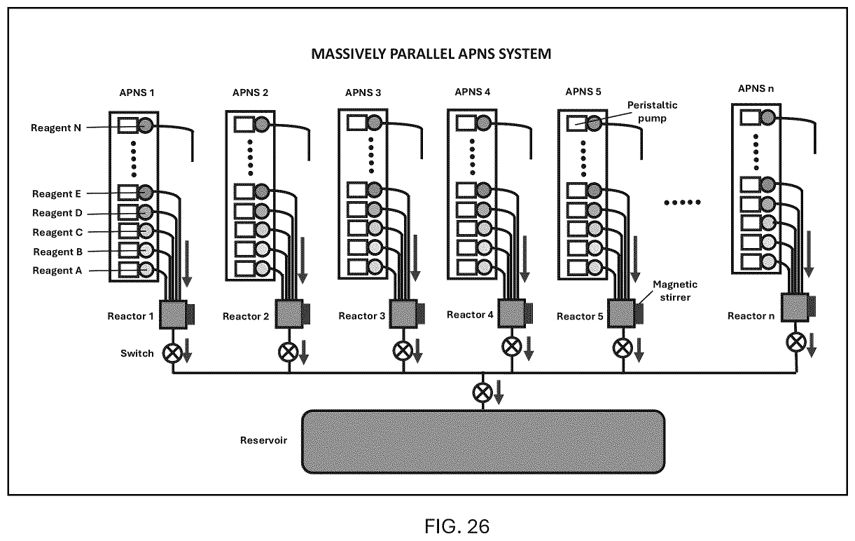

The automated methods and devices provided herein enable production of large quantities of GNS synthesized identically with machine precision. FIG. 26 is a schematic diagram showing an embodiment of a Massively Parallel AC-GNS system that can be used for large-scale production of nanoparticles.

A system is provided for producing plasmonic-active gold nanostars, comprising: (a) at least one reaction vessel; (b) a reagent dispensing module comprising a microcontroller and at least one motor; and (c) software to execute a method comprising adding to a single one of the at least one reaction vessels with rapid mixing, predetermined timing, and in sequential order the following reagents: (i) an acidic water solution, (ii) a gold ion solution, (iii) a plurality of gold nanospheres, (iv) a silver ion solution, and (v) an ascorbic acid solution, to trigger branch formation on a surface of the gold nanospheres, thereby forming plasmonic-active gold nanostars.

In the system, the method can further comprise adding a thiolated polyethylene glycol solution to the formed plasmonic-active gold nanostars.

In the system, the method can further comprise adding a second addition of the silver ion solution to the plasmonic-active gold nanostars, followed by adding an ammonium hydroxide solution to cause a layer of silver to form on a core of the plasmonic-active gold nanostars and to grow outwards to a tip of the branches, thereby forming plasmonic-active bimetallic nanostars.

The Automated Plasmonic Nanoparticle Synthesis (AC-GNS) system for fabricating nanoparticles can greatly contribute to achieving Good Manufacturing Practice (GMP) by improving consistency, quality control, data tracking, safety, and traceability. These factors are essential for ensuring the quality and safety of nanoparticle-based products, particularly in pharmaceutical and biotechnology industries as well as biomedical applications. GMP is a set of guidelines and regulations that ensure the quality, safety, and consistency of products. The advantages of AC-GNS for GMP are the following:
1) Reproducibility and Consistency: Automation ensures that the synthesis process is carried out with reproducibility, consistency, and precision by minimizing human error and variability. This leads to a high degree of consistency and reproducibility in nanoparticle production, a key requirement for GMP compliance.
2) Quality Control: An automated system can continuously monitor and control various parameters such as the timing of reactions, reactant concentrations, temperature, pressure, pH, etc. This real-time monitoring allows for immediate adjustments and reduces the risk of deviations from established quality standards.
3) Prevent or Minimize Contamination Risk: Automation reduces the need for human intervention, which can introduce contaminants into the production process. This reduces the risk of contamination and cross-contamination, a significant concern in GMP.
4) Validation and Qualification: Automated systems can be thoroughly validated (software, hardware, etc.) and qualified to ensure they consistently meet GMP requirements. This includes validation of the entire production process.
5) Scalability: Automated systems can be designed to handle a range of batch sizes and large volumes of products (e.g., in our case a large number of small-volume reactors), making it easier to scale up or down according to demand. This scalability is important for meeting production requirements in compliance with GMP.
6) AI and Machine Learning (ML) techniques are most suitable for automated synthesis systems, which can provide large numbers of products by varying synthesis and experimental parameters. These parameters can be used by AI/ML techniques for improved synthesis.
7) Data Logging and Error Alert & Correction: Automated systems can record, and store data related to the synthesis process, including timestamps and all relevant parameters. This data can be used to provide a comprehensive record of each batch, which is essential for GMP compliance and traceability. Automated systems can be programmed to detect deviations from the desired process parameters and initiate corrective actions or generate alerts.
8) This data logging capability is important for Good Laboratory Practice (GLP), which focuses on the integrity and reliability of data generated during the research, development, and testing phases of products, especially in pharmaceuticals and chemicals. GLP focuses on the quality and accuracy of laboratory results provided by the data logging process.
9) Do No Require Operator or Minimize Operator Training: With automation, there is less reliance on skilled operators, reducing the time and cost of training. This is particularly relevant for GMP compliance since operators must be well-trained and regularly requalified.
10) Improved Safety: Automation can minimize the exposure of personnel to hazardous materials, as the system can handle these potentially toxic substances without human intervention. This enhances the safety of the manufacturing process, which is a critical aspect of GMP.
11) Regulatory Compliance: Many GMP guidelines and regulations require a high level of control and traceability, which an automated system can provide. Automated synthesis systems can be designed and operated in accordance with GMP regulations, facilitating compliance.

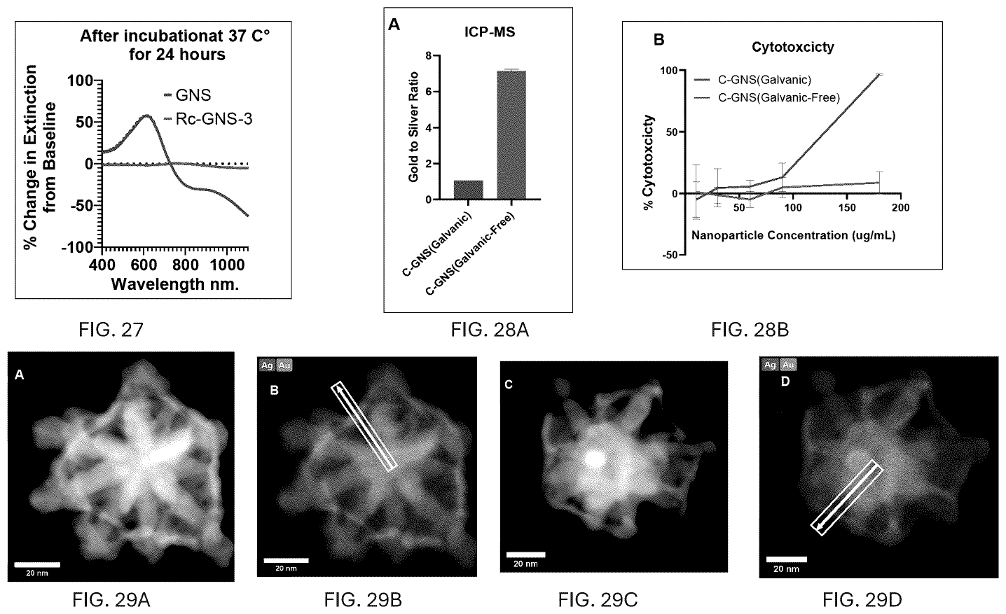

FIG. 27 shows the change in absorption spectrum between older-generation gold nanostar particles (i.e., lacking a cage) and C-GNS particles after being incubated at 37° C. for 24 hours. The shift in absorption in the NIR for the C-GNS particles is significantly less when compared to the gold nanostar particles.

FIG. 28A shows the results of ICP-MS analysis of C-GNS particles prepared via the galvanic replacement and galvanic replacement-free methods, where the galvanic replacement-free method results in a significantly higher gold-to-silver ratio. FIG. 28B shows the results of the cytotoxicity study comparing C-GNS particles prepared with the galvanic replacement method and galvanic replacement-free methods. The C-GNS particles prepared via galvanic replacement were significantly more cytotoxic compared to the C-GNS particles prepared via the galvanic replacement-free method.

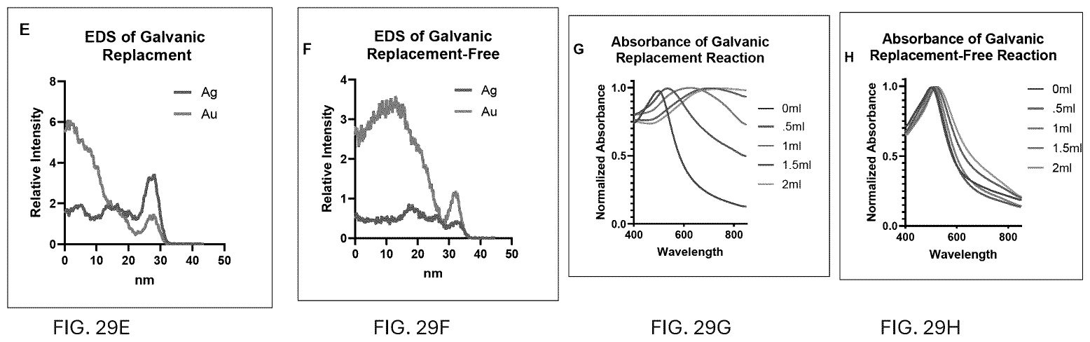

FIGS. 29A-29B show HAADF-STEM and EDS-STEM imaging of C-GNS particles prepared using the galvanic replacement method. FIGS. 29C-29D show C-GNS particles prepared with the galvanic replacement-free method. FIG. 29E shows the quantification of the STEM-EDS image shown in FIG. 29B. FIG. 29F shows the quantification of the STEM-EDS image shown in FIG. 29D. Quantification reveals there is a far higher amount of gold-silver alloy in the shell of the C-GNS particle prepared using galvanic replacement compared to a shell of a C-GNS particle that was prepared via the galvanic replacement-free method. The change in the absorption spectra of C-GNS solutions during the galvanic replacement process is shown in FIG. 29G, and the change in the absorption spectra of C-GNS particles during the galvanic replacement-free method is shown in FIG. 29H.

### Example 1

### Synthesis and Testing of Plasmonic-Active Caged Gold Nanostars

To initiate C-GNS preparation, GNS were synthesized using a surfactant-free method. FIG. 1A contains a representative STEM-HAADF image of a typical GNS used as an initial building block of a C-GNS particle. A layer of silver was then deposited onto the exposed core of the GNS, growing outwards to the branch tips, resulting in a bimetallic nanostar (BNS). A STEM-EDS image showing the overlay of the distribution of silver and gold throughout a representative BNS particle is shown in FIG. 1B. Next, BNS particles were dispersed into a basic PVP solution containing 0.1M ascorbic acid. Then gold chloride was added via a syringe pump to the solution. FIGS. 1C and 1D show STEM-EDS images of the elemental overlay and the gold-only channel from a gold-coated BNS particle. Under these experimental conditions, the rate of gold ion reduction onto the surface of the BNS is greater than that of galvanic replacement. While the galvanic replacement reaction would result in the formation of a hollow cage structure, a by-product of that process would be the formation of a gold-silver alloy as the shell. Therefore, the galvanic replacement-free synthetic route was chosen, which results in the lowest amount of gold-silver alloy formation in the shell of the C-GNS.

Gold-coated BNS particles were washed and redispersed into an acidic 3% hydrogen peroxide solution at 65° C. to etch the golden outer shell and remove the internal silver layer. This method of hollow shell formation uses the original silver shell as a removable scaffold upon which to build the golden outer shell. This etching process results in an unsealed C-GNS. FIG. 1E contains a STEM-EDS image of the gold and silver channel overlay from an unscaled C-GNS, and FIG. 1F contains the corresponding STEM-HAADF image. The high efficiency of C-GNS formation is seen in the STEM-HAADF image in FIG. 1G, where all BNS that enter the galvanic replacement-free gold coating process result in C-GNS. The plot in FIG. 1H contains the normalized absorption spectra of each nanoparticle solution. GNS has a tuneble plasmon resonance in the NIR, depending on branch length and aspect ratio. The maximum absorbance for the representative batch shown here occurs at 736 nm. The addition of silver onto the surface of the GNS results in a dramatic blue shift of the absorbance spectrum, resulting in a peak at 498 nm. A small red shift in peak absorbance to 513 nm is observed once the galvanic replacement-free gold deposition is complete. After silver oxidation and removal via hydrogen peroxide etching, the absorbance of the C-GNS particle solution dramatically redshifted. This resulted in a peak absorbance again at 736 nm. There was a noticeable widening of the absorption spectra between GNS and C-GNS solutions. The wave optics module of COMSOL Multiphysics 6.0 was used to evaluate the structural cause of this widening, with corresponding models for GNS and C-GNS particles (data not shown). The C-GNS model uses the same base particle as the GNS model, adding a 1 nm thick gold shell and all exposed branches with an increased radius of 1 nm. FIG. 1I contains the heat losses spectra for both particle models. This value is the result of integrating the resistive losses of the model at each wavelength with the volume of the model. While the heat losses spectra and the absorption spectra describe different physical phenomena, the heat losses spectra has been shown to provide information about trends seen in absorbance spectra. Points labeled i, ii, and iii in FIG. 1I correspond to FIGS. 1J-1K, respectively. These figures depict the normalized electric field |E|/|Eo| around the nanoparticle model at the given wavelength. The resonance peaks labeled i and ii in FIG. 1I correspond to the tips of the GNS and C-GNS branches shown in FIGS. 1J and 1K. FIG. 1L, which represents the interaction between the C-GNS model and 900 nm light, shows an intense enhancement in the local electric field. The plasmonic coupling between the particle branches and shell supports the peak broadening in the NIR region of the C-GNS absorption spectra.

After successfully developing a method to create a hollow shell around a GNS as described above, further chemical modification and cargo loading of the C-GNS was performed. Unsealed C-GNS from the procedure described herein above, were washed several times in ethanol to remove surface-bound PVP and to allow access to the etched opening along the shell surface. HITC dye was added to the C-GNS ethanol solution as a proof-of-concept study to examine the loading capability of the particles. After mixing overnight to ensure maximal dye diffusion, aliquots of dye-loaded nanoparticle solution were redispersed individually into an aqueous PVP solution and coated with a final layer of gold. Five different molar ratios of ionic gold to nanoparticles in solution were tested ranging from 5×105 to 2.5×106 in increasing increments of 5×105 to examine how the thickness of the golden shell would affect the optical properties of the newly sealed C-GNS. These formulations are referred to herein as AU_1-AU_5, corresponding to their relative increase in molar ratio. After the final coating, the particles were washed several times in ethanol to remove surface-bound PVP and incubated in a 20 uM COOH-PEG5000-SH ethanol solution at 50° C. overnight to ensure complete surface-adsorbed PVP or dye removal.

FIGS. 2A-2C contain STEM-EDS images of representative gold-coated C-GNS from AU_1, AU_3, and AU_5 batches. Below in FIGS. 2D-2F are the corresponding STEM-EDS overlay images. The brightfield STEM image in FIG. 2G provides improved internal nanoparticle contrast as the shell thickness increases compared to HAADF. As the gold layer increases in thickness, the peak in the absorption spectrum blue shifts, while the relative absorbance in the 800 nm range decreases, most noticeably seen in the AU_5 formulation in FIG. 2H. The widening of the branch tips seen in FIGS. 2C and 2F causes this blue shift to occur. SERS measurements of each gold-coated sample were taken to investigate the effect that increasing the thickness of the outer gold layer has on signal intensity. The sample which had received the lowest molar ratio of gold, AU_1, had the lowest SERS signal, shown in FIG. 2I. Samples AU_2,3,4 had very similar intensities, with sample AU_5 having the largest SERS signal.

To confirm that the HITC dye was sealed with the C-GNS and not adsorbed onto the particle surface, aliquots of sealed AU_3 and unsealed C-GNS particles were tested with hydrogen peroxide. First, an aliquot of unsealed C-GNS from the dye-loading solution was centrifuged and resuspended in an aqueous PVP solution so the fluorescence of the unloaded dye would not interfere with subsequent measurements. H2O2 was added to sealed and unsealed C-GNS samples, and SERS measurements were recorded over 1 hour. The resulting spectra are shown in FIG. 2J. Before adding H2O2, the SERS signal of the uncoated CNS sample was much lower than that of the same concentration of sealed C-GNS particles. The unsealed C-GNS may have lost internally diffused dye during centrifugation before being resuspended. After adding H2O2, the SERS signal intensity of both samples decreased. However, the unsealed samples experienced a near-complete signal loss after 15 minutes of exposure. The slow signal decay of the AU_3 sample considering the several ethanol washes and subsequent thiolated Peg functionalization is strong evidence of effectively encapsulated cargo.

To investigate the SERS signal enhancement trends seen in FIG. 2, the wave optics package in COMSOL Multiphysics was used to examine the local electric field enhancement |E|/|Eo| at 785 nm for a wide array of different C-GNS morphologies. Three nanoparticle dimension parameters were varied to encompass the heterogeneity in these C-GNS particles of various gold coating thicknesses. The schematic in FIG. 3A is the upper right quadrant of a C-GNS model cross-section with the swept dimensions indicated with red arrows. Starting from the left, the first arrow indicates the distance between the branch tip and the inner edge of the gold shell, which was simulated at 15 nm, 10 nm, 5 nm, and 1 nm. The second arrow represents the shell thickness, which was swept between 1 and 8 nm in thickness in steps of 1 nm. Finally, the third red arrow in FIG. 3A depicts the branch length, simulated at 25 nm, 30 nm, 35 nm, 40 nm, 45 nm, and 50 nm.

Here, the goal was to examine how these subtle morphological changes affect the overall SERS signal intensity of the C-GNS particles. Only the interior of the particle model was considered for heat losses integration because this is the only dye-containing region. The internal heat losses result of all 192 simulated C-GNS model variants are shown in the scatter plot in FIG. 3B. The x, y, and z axes are labeled with the swept parameters, and the color scale corresponds to the magnitude of the heat losses value. The domain of swept dimensions where the greatest degree of local electric field enhancement was observed was in particles with a branch length between 30 and 45 nm, an inner shell edge-to-branch tip distance between 5 and 15 nm, and a gold shell thickness between 4 and 8 nm. This range closely matches all particle dimensions observed experimentally and supports the SERS signal intensity measures in FIG. 2I. Within this specified range, no geometry displayed a high internal heat loss value with a shell thickness between 1 and 3 nm. This shell thickness range corresponds experimentally to the AU_1 formulation, producing a significantly lower SERS signal than the other 4 sample morphologies. Evidence of further agreement between experimental and theoretical results can be seen between the subtle increase in SERS signal in AU_2-AU_5 and the corresponding increase of heat loss values of models with a shell thickness between 4 and 8 nm in this range. The internal heat loss value for models with branch lengths 30, 35, 40, and 45 nm and branch tip to inner shell edge distances of 15, 10, and 5 nm were summed as a function of shell thickness. Here, summed heat losses values for these 12 different models increased rapidly from 1 to 4 nm in shell thickness, at which point they leveled off from 5 to 8 nm shell thickness. This supports the relatively small increase in the SERS signal between AU-2 and AU_5. The model shown in FIG. 3C produced the highest internal heat losses value, with a branch length of 35 nm, a branch tip to inner shell edge distance of 5 nm, and a shell thickness of 5 nm. FIGS. 3D and 3E show the normalized electric field |E|/|Eo| for the entire particle model and only the interior water cavity. The area of greatest enhancement occurs in the ring-shaped region at the junction of the branch and shell, seen clearly in FIG. 3E, with a maximum |E|/|Eo| value of 234.6 V/m. FIG. 3F contains the interior water cavity's cross-section, illustrating significant enhancement propagating along the branch. The |E|/|Eo| map of the models which generated the greatest internal heat losses values with a branch tip to inner shell edge distance of 5, 10, and 15 nm are included in FIG. 3G to highlight how a variety of morphologies can generate a substantial field enhancement at 785 nm. These particle models generate maximum |E|/|Eo| values of 234.6, 216.1, and 203.3 V/m, respectively.

Finally, the utility of gold-coated C-GNS as theranostic nanoparticles for photothermal therapy was investigated. In the following studies, the AU_2 formulation was used because it produced a significant SERS signal while not dramatically blueshifting the absorption spectrum of the particle solutions. Particles were first characterized using an agarose gel phantom (data not shown). FIG. 4A shows the total emission of the phantom when excited with 785 nm light. The peak absorption wavelength for this fluorescent dye is 740 nm, so while further increasing the SERS signal of these particles via resonant Raman, there is also an appreciable fluorescent background. A liquid crystal tunable filter with a 0.75 nm FWHM bandpass was then used to directly image the phantom at 820, 822, and 824 nm. The resulting direct images at these wavelengths are shown in FIG. 4B in ascending order. The reference spectra of HITC dye in C-GNS is shown in FIG. 4C, where the labels I, ii, and iii correspond to the bottom, middle and top slices of FIG. 4B. While there is a significant signal due to the background fluorescence of the dye, there is a noticeable increase in the signal of the center slice in FIG. 4B.

To evaluate the performance of the gold-coated C-GNS in vivo, 1.5 mg of nanoparticles were administered via retroorbital injection to albino B6 mice with a bladder cancer flank tumor. FIG. 4D shows a mouse 24 hours after nanoparticle injection; the tumor is in the highlighted region. FIG. 4E is a bright field image of the tumor region through the lab-built microscope system, and FIG. 4F shows the hyperspectral image of the same region at 822 nm. The size of the C-GNS particles allows for passive accumulation within the tumor microenvironment. The high similarity between the bright field and hyperspectral images demonstrates this. The HITC dye encapsulated within the hollow region of the C-GNS enabled the localization of the particles within the animal. The tumor region of each mouse was then irradiated with 1064 nm light with a power density of 450 mW/cm2. The bright field and IR camera images of the photothermal treatment of a mouse that had received C-GNS are shown in FIGS. 4G and 4H. This marked temperature increase is caused by the local increase in NIR absorption generated by the particles. A total of 6 mice were treated with 450 mW/cm2 1064 nm light, three received an injection of 1.5 mg C-GNS, and three did not. The temperature results of a hypodermic thermocouple placed into the center of the tumor are shown as a function of time for each mouse in FIG. 4I. Measurements began 30 seconds before turning on the laser, they continued for another 9 minutes and 30 seconds, at which point the thermocouple was removed, and an image with the thermal camera was taken at the 10-minute mark of laser treatment. The maximum values of each thermal camera measurement are shown in FIG. 4J. A sharp increase in temperature is seen in thermocouple data upon laser activation for all animals which received C-GNS. The average maximum temperature value seen for the group which received C-GNS was 51.4° C., whereas the average of the control group was 38.1° C. The unit used to evaluate clinical hyperthermia is cumulative effective minutes (CEM) at 43º° C. over 90% of the tumor region. Irreversible tissue damage begins to occur at a value of 10 CEM4390. For example, if 90% of the tumor volume were 50° C. for 1 minute, the equivalent CEM4390 value would equal 128, far exceeding the threshold to induce cellular damage. High-resolution temperature mapping is impossible with thermocouples and a thermal camera; therefore, the thermal doses achieved in this study could not be estimated.

In addition to coating the C-GNSs with gold to seal them, the C-GNSs can also be sealed with a coating of silver. FIG. 14 shows how the C-GNSs were sealed with variable amounts of silver, effecting the optical absorption and plasmonic enhancement of the particles. From left to right, the top row (FIGS. 14A-C) contains STEM-EDS images of C-GNS particles coated with an increasing thickness of silver. The subsequent change in the particle absorption spectra is shown in FIGS. 14D-14F. C-GNS particles were filled and sealed with HITC dye, and their SERS spectra as a function of silver thickness is shown in FIGS. 14G and 14H. The SERS signal increases with silver shell thickness.

### Example 2

### Automated Method, Device, and System for Gold Nanostar and Bimetallic Nanostar Synthesis

An automated method, device, and system was developed to allow for GNS synthesis with greatly reduced batch-to-batch differences.

The schematic diagram shown in FIG. 21A outlines the active components of the GNS synthesis device used to add the necessary reagents for GNS synthesis sequentially with predetermined timing steps to a single mixing container. An Arduino Mega microcontroller was used for user input, which in turn controls a total of 6 motors that were used to dispense different reagents necessary for GNS synthesis. All volumes and timing between reagent injections were assigned for each run, allowing for the precise control of synthetic conditions. By adjusting these parameters, the optical properties of the GNS were tuned to fit specific application needs.

FIG. 21B illustrates the synthetic steps that resulted in the formation of GNS particles. For a single batch of GNS, 100 mL of deionized water pH 3 was added to a rapidly mixing flask. Gold chloride solution, followed by gold nanospheres, silver nitrate, and ascorbic acid, was quickly added to trigger branch formation on the surface of the gold nanospheres, resulting in the GNS morphology. The photograph in FIG. 21C shows the GNS synthesis device immediately after producing a batch of nanoparticles. On the left side of the image, 5 50 mL conical tubes can be seen, which contain gold chloride, gold nanosphere solution, silver nitrate, ascorbic acid, and thiolated PEG solution, respectively. A large container containing a dilute hydrochloric acid solution is out of view. All controlling circuitry is housed in the lower section of the GNS synthesis device housing. All peristaltic pumps were chosen for incorporation into the device to ensure no contamination from the pump head. For all 6 motors, one end of the tubing is placed into the reagent reservoir, and the other is fitted with a pipette tip and inserted into the injection head, which is fitted to the neck of the flask. The open-ended design allows for the easy reloading of reagents. This injection head ensures no contact between individual injection channels and all injection channels are oriented vertically directly at the mixing solution below.

In the conceptualization and construction of this device, several variables were identified that may significantly impact the final morphology of GNS particles. These variables include the reaction volume, the quantities of all 5 reagents necessary to achieve stable GNS, the timing between all injection steps, the solution's mixing speed, and the injection volume variability. Therefore, before attempting GNS synthesis, the injection volume accuracy and precision of the device were measured. It was determined that the minimum injection volume of the device to produce particles on this scale would be 1 mL. FIG. 22A shows the volumes of 30 injections in a row, averaging 1001 uL with a standard deviation of 18.26 uL, or 1.8%. With this encouraging result, the next parameter examined was the set speed of the mixing plate used in the reaction. For this initial test, arbitrary device settings were selected that would result in successful GNS synthesis. The reagent addition settings were as follows: 100 mL of dilute acid solution, 1 second pause, 4 ml of gold chloride solution, 1-second pause, 1 mL of 10 nM 12 nm gold spheres, 2 second pause, 1 mL of 5 mM silver nitrate, 5-second pause, 1 mL of 0.1M ascorbic acid, 20-second pause, 1 mL of 5 mg/mL SH-PEG5000 solution. The mixing speed of each run ranged from 100 rpm to 1500 rpm in steps of 200 rpm. The absorption spectra of all GNS solutions produced as a result of this mixing speed study are shown in FIG. 22B. At the lowest speed, the absorption peak is the broadest, indicating the formation of highly heterogeneous GNS in solution. As the mixing speed increases from 300 to 1300 rpm, the absorbance spectra sharpen, and the peak absorption value blue shifts from 1028 nm to 882 nm. At the highest mixing speed, 1500 rpm, the absorbance spectra dramatically narrow, and the peak absorption value further blue shifts to 814 nm. This noticeable peak narrowing indicates an increase in the uniformity of GNS particles within the solution relative to other prepared batches. Plasmonic nanoparticles are generally used in applications like photothermal therapy or SERS sensing that use a single input/excitation wavelength. Therefore, if there is a large degree of homogeneity between nanoparticles in the solution, the solution will interact more strongly with the input light than a solution containing more heterogeneous nanoparticles. Based on these findings, a mixing speed of 1500 rpm was used in all subsequent reactions.

To investigate the tunability of the surfactant-free GNS platform using automated injected volumes and timings, three different base GNS morphologies were tested. These morphologies are titled s10, s25, and s50, where the number refers to the final micromolar concentration of silver nitrate in the GNS solution. For each of these base morphologies, the amount of gold chloride solution added was varied between 3 mL and 5 mL in steps of 0.5 mL. All volumes and timings were held constant and were the same as what was used in the mixing speed study. The resulting absorption spectra of all combinations are shown in FIGS. 22C-22E. Clear trends appear for all base GNS morphologies. Beginning with s10, as the amount of gold chloride in the solution was increased, the absorbance of the solution increased, the main peak value blue shifted from 782 nm to 696 nm, and the peak narrowed. Also, a secondary peak at 1046 nm rose notably when 4.5 mL and 5 mL of gold chloride was added to the solution. A similar trend was seen in the s25 base morphology as well, as the amount of gold chloride increased, the peak absorption value increased, and there was a blue shift from 1030 nm to 850 nm while the peak also narrowed. In the s50 base morphology, the peak absorbance value increased as more gold chloride was added to the solution, however, the value red shifted from 730 nm to 988 nm, while the peak was narrowing. By altering the amounts of input parameters, the optical therapeutic window can be effectively spaned by altering the base GNS morphology with the amount of silver introduced into the solution and the amount of gold chloride added, which controls the GNS branch formation.

To test the repeatability of the nanoparticle synthesis platform, 10 identical batches of S50 GNS particles prepared with 5 mL of gold chloride were synthesized, and with the average absorption spectrum with error bars included shown in FIG. 22 F. The average maximum absorbance value of all 10 batches was 983.8 nm with a standard deviation of 9.1 nm. All injection amounts were held constant to investigate the effects of the specific timing of reagent addition. In contrast, the timing between the addition of silver nitrate and ascorbic acid was varied between 1 second, 5 seconds, 15 seconds, and 30 seconds, and all batches were prepared in triplicate. The resulting absorbance spectra are shown in FIG. 22F, where there is a high degree of similarity between the batches with injection pauses of 1, 5, and 15 seconds. The batches with a pause of 30 seconds are blue shifted in comparison. Importantly, regardless of the duration of the reagent injection pause, there was almost no variation in the absorbance spectra of replicate batches. To confirm that a noticeable change in particle morphology is the cause of the changes in absorbance spectrum seen in FIGS. 22C-22E, HAADF STEM images of s10 particles prepared with 5 mL of gold chloride and s50 particles prepared with 5 mL of gold chloride are shown in FIGS. 22G and 22H, respectively.

FIG. 23 shows that with the addition of a modular motor unit, the GNS synthesis device can be converted into an automatic synthesis machine for bimetallic nanostars. For these particles, the underlying chemistry shares many similarities with GNS synthesis, however, instead of adding SH-PEG5000 as the final step of the reaction, silver nitrate is added for a second time, followed by a dilute ammonium hydroxide solution. These differences cause a layer of silver to form on the core of the GNS particle and grow outwards to the nanoparticle branch tips. The interaction of these two plasmonic active metals leads to the development of another highly tunable nanoplasmonic platform. The attractive properties of these bimetallic nanostar particles (BNS) can be used to provide signal enhancement for SERS biosensing applications.

FIGS. 24A-24F show the absorbance spectra of the different base bimetallic GNS morphologies as the amount of silver added was increased. FIGS. 24G-24I are STEM-EDS images of S25_5 mL_dot25Ag particles, S25_5 mL_1dot25Ag particles, and S25_5 mL_2Ag particles, respectively. To investigate the tunability of these BNS particles, the S25 and S50 base nanostar morphologies were considered due to their contradictory changes in peak absorbance as the amount of gold per particle increased. For both the S25 and S50 base, GNS prepared with the addition of 3 ml, 4 mL, and 5 mL gold chloride were synthesized and coated with varying amounts of silver which ranged from adding 250 μL of 50 mM silver nitrate to 2 mL 50 mM silver nitrate, resulting in a total of 56 different BNS formulations. Clear trends emerged in the changes in absorption spectra as the amount of silver coated onto the surface of the GNS increased. For all base nanostar formulations, the maximum absorption value blue shifted and increased in magnitude as the amount of silver was increased. For both S25 and S50 base morphologies, those prepared with 3 mL gold chloride blue shifted to a greater extent compared to their 5 mL gold chloride counterparts. STEM-EDS imaging was performed to observe the changes in nanoparticle morphology as varying amounts of silver were reduced onto the surface of the GNS.

To assess the performance of these nanoparticles, aliquots from each batch were combined with a p-mercaptobenzoic acid solution for a final concentration of 10 uM. The SERS spectra of each aliquot was recorded using both 633 nm and 785 nm lab-built systems. With this reproducible plasmonic nanoparticle synthesis platform, the nanoparticle morphology can be fine-tuned to generate the greatest degree of signal enhancement. FIG. 25A shows the SERS spectra of S25_5 mL particles with a silver addition amount ranging from 0.25 mL to 2 mL when labeled with p-MBA. FIGS. 25B-25E are graphs showing the peak height at 1586 cm-1 for all synthesized formulations of BNS particles.

The automated methods and devices provided herein enable production of large quantities of GNS-based nanoparticles synthesized identically with machine precision.

One skilled in the art will readily appreciate that the present disclosure is well adapted to carry out the objects and obtain the ends and advantages mentioned, as well as those inherent therein. The disclosure described herein as representative of preferred embodiments, is exemplary, and is not intended as a limitation on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. It will be understood that, unless otherwise stated, reference to any document herein does not constitute an admission that any of these documents form part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A plasmonic-active caged gold nanostar, comprising:
one or more plasmonic-active caged gold nanostar particles having a hollow gold shell surrounding an essentially spherical core and a plurality of gold branches protruding out from the core, wherein a thickness of the gold shell is essentially uniform and tunable, and wherein the one or more plasmonic-active caged gold nanostar particles:
are essentially free of surfactant,
have a tunable plasmon resonance ranging from the visible to the near-infrared (NIR) spectral range, and
optionally, comprise one or more compounds encapsulated within the hollow gold shell.

2. The plasmonic-active caged gold nanostar of claim 1, wherein a length of the branches ranges from 10-50 nm, a distance from an inner edge of the shell to a tip of the branches ranges from 1-20 nm, and the thickness of the gold shell ranges from 1-15 nm.

3. The plasmonic-active caged gold nanostar of claim 1, wherein the plasmonic-active caged gold nanostar is produced using a galvanic replacement-free reaction method and has a reduced amount of a gold-silver alloy in the gold shell relative to a plasmonic active caged gold nanostar produced using a galvanic replacement reaction method.

4. The plasmonic-active caged gold nanostar of claim 1, wherein the one or more plasmonic-active caged gold nanostar particles generates a greater local electric field enhancement ([E]/[E0]) from the visible to the NIR spectral range relative to a plasmonic-active gold nanostar of a similar diameter that lacks the hollow gold shell.

5. The plasmonic-active caged gold nanostar of claim 1, wherein the plasmonic-active caged gold nanostar retains a greater degree of NIR absorption intensity relative to a plasmonic-active gold nanostar that lacks the hollow gold shell.

6. The plasmonic-active caged gold nanostar of claim 5, wherein the greater degree of NIR absorption intensity is at least 20%.

7. The plasmonic-active caged gold nanostar of claim 1, wherein a diameter of the one or more plasmonic-active caged gold nanostar particles is 300 nm or less.

8. The plasmonic-active caged gold nanostar of claim 1, wherein the one or more plasmonic-active caged gold nanostar particles further comprises an attached bioreceptor comprising one or a combination of an antibody, a nanobody, an antigen, a peptide, an aptamer, a nucleic acid, an enzyme, a molecular sentinel (MS), or an inverse molecular sentinel (iMS).

9. The plasmonic-active caged gold nanostar of claim 5, wherein the bioreceptor comprises an optical reporter.

10. The plasmonic-active caged gold nanostar of claim 1, wherein the one or more compounds comprises a dye or a drug.

11. A method of making plasmonic-active caged gold nanostars, comprising:
dispersing one or more bimetallic nanostars into a basic polyvinylpyrrolidone solution, wherein the one or more bimetallic nanostars comprise a gold nanostar having a spherical core, a plurality of branches protruding out from the core, and a layer of silver on the core and extending outward onto the branches, wherein a tip of the branches are exposed gold; and
performing one of the following two reactions:
i. exchanging gold ions with silver atoms of the bimetallic nanostar in a galvanic replacement reaction, or
ii. depositing gold ions to coat a silver surface of the bimetallic nanostars with a layer of gold in a galvanic replacement-free reaction, thereby forming one or more gold-coated bimetallic nanostars, and redispersing the one or more gold-coated bimetallic nanostars into a second solution of hydrogen peroxide to etch the gold layer and remove the silver,
thereby forming one or more plasmonic-active caged gold nanostars having a hollow gold shell surrounding the essentially spherical core.

12. The method of claim 11, further comprising:
optionally, mixing the one or more plasmonic-active caged gold nanostars in a third solution with a compound to be loaded into the hollow gold shell, and
dispersing the plasmonic-active caged gold nanostars in a fourth solution and adding gold ions to coat the plasmonic-active caged gold nanostars with a final layer of gold, thereby forming plasmonic-active gold-coated caged gold nanostars and, optionally, having the compound encapsulated within the hollow gold shell,
wherein a thickness of the gold shell is essentially uniform and tunable, and wherein the plasmonic-active gold-coated caged gold nanostars have a tunable plasmon resonance from the visible to the near-infrared (NIR) spectral range.

13. The method of claim 11, further comprising:
optionally, mixing the one or more plasmonic-active caged gold nanostars in a third solution with a compound to be loaded into the hollow gold shell, and
dispersing the plasmonic-active caged gold nanostars in a fourth solution and adding silver ions to coat the plasmonic-active caged gold nanostars with a layer of silver, thereby forming silver-coated plasmonic-active caged gold nanostars and, optionally, having the compound encapsulated within the gold and silver-coated hollow shell,
wherein a thickness of the silver-coated gold shell is essentially uniform and tunable, and wherein the plasmonic-active silver-coated caged gold nanostars have a tunable plasmon resonance from the visible to the near-infrared (NIR) spectral range.

14. The method of claim 11, further comprising:
forming the one or more bimetallic nanostars by depositing a layer of silver onto an exposed core of one or more gold nanostars, each nanostar having a plurality of branches protruding out from the core, wherein the layer of silver grows outward onto the branches and leaves a tip of the branches exposed, thereby forming the one or more bimetallic nanostars.

15. The method of claim 11, wherein the plasmonic-active caged gold nanostars are essentially free of surfactant.

16. The method of claim 11, wherein the plasmonic-active caged gold nanostars are formed according to reaction (ii) and have a reduced amount of gold-silver alloy in the gold shell compared to plasmonic-active caged gold nanostars formed according to reaction (i).

17. The method of claim 11, wherein the dispersing and exchanging steps, or the dispersing, depositing, and redispersing steps are automated and occur in a single reaction vessel with rapid mixing and predetermined timing.

18. A method for in vitro detection of a target of interest, comprising:
contacting one or more plasmonic-active caged gold nanostar particles of claim 1 with a sample, wherein the one or more plasmonic-active caged gold nanostar particles further comprises a bioreceptor for one or more of a cell, a nucleic acid, or a protein target of interest and, optionally, an optical reporter; and
detecting an optical signal from one or both of the optical reporter and the one or more compounds encapsulated within the hollow gold shell.

19. The method of claim 18, wherein the bioreceptor comprises a nucleotide sequence, an aptamer, an antibody, an enzyme, or a cell-based receptor.

20. The method of claim 19, wherein the bioreceptor comprises:
a stem-loop nucleic acid probe comprising a sequence that forms a stem loop, a first end attached to the one or more plasmonic-active caged gold nanostar particles, and a second end labeled with the optical reporter, and
an unlabeled capture placeholder nucleic acid strand comprising a first region complementary to a sequence in a nucleic acid target of interest and a second region complementary to the sequence that forms a stem-loop or a portion thereof, wherein the second region is shorter than and overlapping the first region,
wherein in the presence of the nucleic acid target the placeholder nucleic acid strand binds to the sequence in the nucleic acid target of interest and the stem loop closes, thereby inducing the optical signal for detection.

21. The method of claim 20, wherein the nucleic acid target of interest comprises a microRNA, a small noncoding RNA, an mRNA, or a DNA sequence.

22. The method of claim 18, wherein the optical signal is a Raman signal or surface-enhanced Raman scattering (SERS) signal.

23. The method of claim 18, wherein the optical reporter is selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, crystal violet, a fluorescence label, and absorbance label.
